(19) United States
(12) Patent Application Publication
(10) Pub. No.: US 2025/0197373 A1
(43) Pub. Date: Jun. 19, 2025

(54) SMALL MOLECULE COMPOUND OR SALT THEREOF OR SOLVATE OF THEM, GUANINE NUCLEOTIDE-BINDING PROTEIN G(Q) SUBUNIT ALPHA (GNAQ) INHIBITOR CONTAINING THE SAME AND METHOD FOR TREATING AND/OR PREVENTING CANCER

(71) Applicant: INDUSTRIAL TECHNOLOGY RESEARCH INSTITUTE, Hsinchu (TW)

(72) Inventors: Wan-Ru CHEN, Hsinchu City (TW); Chih-Hung CHEN, Zhudong Township (TW); Yow-Lone CHANG, Hsinchu City (TW); Shu-Feng CHEN, Hsinchu City (TW); Hsiang-Ching WANG, Hsinchu City (TW); Shu-Ling WANG, Hsinchu City (TW); Yen-Ju LIN, Taichung City (TW); Ming-Hsi WU, Taipei City (TW)

(73) Assignee: INDUSTRIAL TECHNOLOGY RESEARCH INSTITUTE, Hsinchu (TW)

(21) Appl. No.: 18/964,262

(22) Filed: Nov. 29, 2024

Related U.S. Application Data
(60) Provisional application No. 63/604,563, filed on Nov. 30, 2023.

(51) Int. Cl. C07D 401/14; A61K 31/444; A61K 31/505; A61K 31/506; A61P 35/00; C07D 213/75; C07D 239/26; C07D 401/04; C07D 401/12

(52) U.S. Cl. CPC C07D 401/14; A61K 31/444; A61K 31/505; A61K 31/506; A61P 35/00; C07D 213/75; C07D 239/26; C07D 401/04; C07D 401/12

(57) ABSTRACT

A small molecule compound or salt thereof or solvates of them is provided, wherein the small molecule compound comprises a compound represented by Formula (I) as shown below:

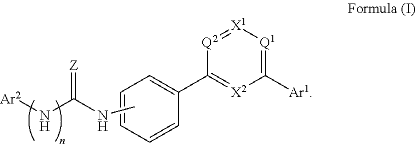

### CROSS REFERENCE TO RELATED APPLICATIONS

This application claims the benefit of U.S. Provisional Application No. 63/604,563, filed on Nov. 30, 2023, the entirety of which is incorporated by reference herein.

### TECHNICAL FIELD

The present disclosure relates to a novel small molecule compound, and in particular it relates to a small molecule compound or salt thereof or solvates of them, a guanine nucleotide-binding protein G(q) subunit alpha (GNAQ) inhibitor and pharmaceutical composition containing the same, and use thereof.

### BACKGROUND

Guanine nucleotide-binding protein G(q) subunit alpha (GNAQ) is one of the most critical regulators and converters of various G protein-coupled receptors (GPCRs). GNAQ mainly affects downstream signaling through the MAPK signaling pathway.

Tumorogenesis is related to the abnormal expression and activity of GPCRs and G proteins related thereto. Oncogenic mutations of GNAQ have been found in many types of tumors. For example, a common GNAQ mutation in uveal melanoma is Q209L while a common GNAQ mutation in NK/T cell lymphoma and liver cancer is T96S. In Chinese specimen data, it has been found for the first time that GNAQ in more than 18% of breast cancer samples carries the T96S mutation, and GNAQ with the T96S mutation (GNAQ (T96S)) is more capable of activating downstream signaling compared to wild-type GNAQ (GNAQ (WT)).

At present, very few compounds have been reported to have GNAQ inhibitory activity, and there is still great room for improvement. Therefore, there is still an urgent need to develop a GNAQ inhibitor and/or drug that can effectively inhibit GNAQ and is not affected by the specific mutation point T96S of GNAQ.

### SUMMARY

The present disclosure provides a small molecule compound or salt thereof or solvates of them, wherein the small molecule compound comprises a compound represented by Formula (I) as shown below:

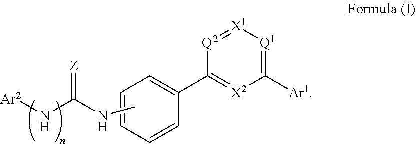

In Formula (I), Q¹ is CR¹ or N, Q² is CR² or N, X¹ is CR³ or N, and X² is CR⁴ or N, wherein R¹ and R² are independently H, alkyl, alkylhalo or halogen, R³ and R⁴ are independently H or alkyl, and when Q¹ and X² are both N, Q² is CR², and X¹ is CR³, when Q² and X² are both N, Q¹ is CR¹, and X¹ is CR³, when Q¹ and Q² are both N, X¹ is CR³, and X² is CR⁴. Furthermore, in Formula (I), Z is O or S, and n is 0 or 1. Moreover, in Formula (I), Ar¹ and Ar² are independently unsubstituted or substituted aryl or heteroaryl, and the substituted aryl or heteroaryl is substituted by at least one substituent selected from a group consisting of: halogen, cyano, NH₂, NHR⁵, NR⁶R⁷, NO₂, alkyl, OH, CF₃, —O-alkyl, —SO₂— alkyl, —SO₂N-alkyl, —S-alkyl and (C₁-C₄)alkoxy(C₁-C₄)alkyl, wherein R⁵ and R⁶ are independently alkyl, and R⁶ and R⁷ are the same, and the alkyl can be unsubstituted or substituted straight-chain alkyl or branched-chain alkyl, and the substituted straight-chain alkyl or branched-chain alkyl is substituted by at least one substituent selected from a group consisting of halogen, oxygen and amino.

Moreover, the present disclosure provides a guanine nucleotide-binding protein G(q) subunit alpha (GNAQ) inhibitor, comprising: the aforementioned small molecule compound or salt thereof or solvates of them.

Furthermore, the present disclosure also provides a use of the foregoing small molecule compound or salt thereof or solvates of them as a GNAQ inhibitor.

The present disclosure additionally provides a use of the aforementioned small molecule compound or salt thereof or solvates of them in the manufacture of a GNAQ inhibitor.

The present disclosure further provides a pharmaceutical composition, comprising: the aforementioned small molecule compound or salt thereof or solvates of them.

In addition, the present disclosure also provides a use of the aforementioned small molecule compound or salt thereof or solvates of them in the manufacture of a medicament.

The present disclosure may further provide a pharmaceutical composition for treating and/or preventing a GNAQ-related disease and/or symptom, comprising: the aforementioned small molecule compound or salt thereof or solvates of them.

The present disclosure also provides a use of the aforementioned small molecule compound or salt thereof or solvates of them in the manufacture of a medicament for treating and/or preventing a GNAQ-related disease and/or symptom.

The present disclosure further provides a method for treating and/or preventing a GNAQ-related disease and/or symptom, comprising: administering the aforementioned small molecule compound or salt thereof or solvates of them, or the aforementioned pharmaceutical composition for treating and/or preventing a GNAQ-related disease and/or symptom, to a subject in need thereof.

In addition, the present disclosure further provides a pharmaceutical composition for treating and/or preventing a cancer, comprising: the aforementioned small molecule compound or salt thereof or solvates of them.

The present disclosure also may provide a use of the aforementioned small molecule compound or salt thereof or solvates of them in the manufacture of a medicament for treating and/or preventing a cancer.

The present disclosure further provides a method for treating and/or preventing a cancer, comprising: administering the aforementioned small molecule compound or salt thereof or solvates of them, or the aforementioned pharmaceutical composition for treating and/or preventing a cancer, to a subject in need thereof.

A detailed description is given in the following embodiments.

### BRIEF DESCRIPTION OF THE DRAWINGS

None.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details.

### Definition

In the present disclosure, the term “and/or” is meant to contain all suitable combinations of “and” and “or”. For example, “A and/or B” contains the following 3 variations: (i) A; (ii) B; (iii) A and B. Also, for example, “A, B and/or C” contains the following 7 variations: (i) A; (ii) B; (iii) C; (iv) A and B; (v) A and C; (vi) B and C; and (vii) A, B and C.

In the present disclosure, the term “alkyl” refers to a monovalent group derived from aliphatic hydrocarbons by removing hydrogen atom. The skeleton structure does not contain a heteroatom (atom other than carbon and hydrogen atoms) or unsaturated carbon-carbon bonds, and has a hydrocarbon group or a partial collection of hydrocarbon structures containing hydrogen and carbon atoms. Alkyl may include straight-chain alkyl and may include branched-chain alkyl groups. Alkyl may be alkyl with 1-24 carbon atoms (C₁-C₂₄, the following “Cₚ-C_{q}” means that the number of carbon atoms is p-q), such as C₁-C₂₀ alkyl, C₁-C₁₀, C₁-C₆ alkyl, etc., but it is not limited thereto. Specific examples of alkyl include methyl, ethyl, n-propyl, i-propyl, n-butyl, s-butyl, t-butyl, isobutyl (2-methylpropyl), n-pentyl, s-pentyl (1-methylbutyl), t-pentyl (1,1-dimethylpropyl), neopentyl (2,2-dimethylpropyl), isopentyl (3-methylbutyl), 3-pentyl (1-ethylpropyl), 1,2-dimethylpropyl, 2-methylbutyl, n-hexyl, 1,1,2-trimethylpropyl, 1,2,2-trimethylpropyl, 1,1,2,2-tetramethylpropyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, etc.

In the present disclosure, the term “alkoxy” denotes a group resulting from the linkage of an “alkyl” as defined above with an oxygen atom, such as a C₁-C₂₄ alkoxy. Examples of alkoxy may include, methoxy, ethoxy, 1-propoxy, 2-propoxy, n-butoxy, i-butoxy, s-butoxy, t-butoxy, pentoxy, 3-methylbutoxy, etc., but are not limited thereto.

Moreover, in the present disclosure, the term “aryl” means a monovalent aromatic hydrocarbon ring, such as a C₆-C₁₀ aryl group. Specific examples of the aryl include phenyl, naphthyl (such as 1-naphthyl, 2-naphthyl), etc. In the present disclosure, the aryl may also include a bicyclic aryl in which the foregoing aromatic hydrocarbon ring is condensed with other saturated or unsaturated ring, for example, an aryl with a condensed ring structure in which the aromatic hydrocarbon ring is a benzene ring and the saturated ring is a 5-membered, 6-membered or 7-membered saturated hydrocarbon ring or saturated heterocyclic ring, such as indanyl, 1,2,3,4-tetrahydronaphthyl, 2,3-dihydrobenzofuran, etc.

In the present disclosure, the term “heteroaryl” refers to an aromatic cyclic monovalent group containing 1-5 heteroatoms in addition to carbon atoms. The ring may be a single ring, may be a condensed ring with other ring, or may be partially saturated. The number of atoms constituting the ring may be 5-10 (5-10 membered heteroaryl), such as 5-7 (5-7 membered heteroaryl), but is not limited thereto. Examples of heteroaryl groups may include, but are not limited to, furyl, thienyl, pyrrolyl, imidazolyl, pyrazolyl, thiazolyl, isothiazolyl, oxazolyl, isooxazolyl, oxadiazolyl, thiadiazolyl, triazolyl, tetrazolyl, pyridyl, pyrimidinyl, pyridazinyl, pyrazinyl, triazinyl, benzofuranyl, benzothienyl, benzothiadiazolyl, benzothiazolyl, benzooxazolyl, benzooxadiazolyl, benzimidazolyl, indolyl, isoindolyl, indazolyl, quinolyl, isoquinolinyl, cinnolinyl, quinazolinyl, quinoxalinyl, benzodioxolyl, indolizinyl, imidazopyridyl, etc.

The term “amino” in the present specification denotes —NH₂ in a narrow sense and —NRR′ in a broad sense. R and R′ may be independently selected from hydrogen, alkyl, alkenyl, alkynyl, cycloalkyl, heterocyclyl, aryl, or heteroaryl, or R and R′ may together with the nitrogen atoms bonded thereto form a ring. The amino may be such as —NH₂, mono-C₁-C₆ alkylamino, di-C₁-C₆ alkylamino, 4-8 membered cyclic amino, etc.

### DESCRIPTION

The present disclosure may provide a novel small molecule compound, and may simultaneously provide salts of the novel small molecule compound, and provide solvates of the novel small molecule compound or solvates of the salts of the novel small molecule compound.

The small molecule compound or salt thereof or solvates of them of the present disclosure may have an effect of inhibiting guanine nucleotide-binding protein G(q) subunit alpha (GNAQ), an effect of treating and/or preventing a disease and/or symptom, etc., but they are not limited thereto. Examples of the aforementioned disease and/or symptom may include, but are not limited to, a cancer, Sturge-Weber syndrome, Klippel-Trenaunay syndrome, port wine stain, etc. Examples of the foregoing cancer may include, but are not limited to, melanoma, lymphoma, liver cancer, breast cancer, etc. The above-mentioned melanoma may include uveal melanoma, but it is not limited thereto. The above-mentioned lymphoma may include, but is not limited to, NK/T cell lymphoma. In addition, the breast cancer mentioned above may include mammary gland cancer, triple-negative breast cancer, etc., but it is not limited thereto.

The foregoing small molecule compound of the present disclosure may comprise a compound represented by Formula (I) as shown below, but it is not limited thereto:

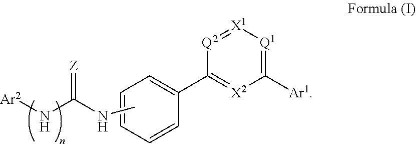

In Formula (I) shown above, Q¹ may be CR¹ or N, Q² may be CR² or N, X¹ may be CR³ or N, and X² may be CR⁴ or N. Moreover, when Q¹ and X² are both N, Q² is CR², and X¹ is CR³H, when Q² and X² are both N, Q¹ is CR¹, and X¹ is CR³, when Q¹ and Q² are both N, X¹ is CR³, and X² is CR⁴.

The R¹ and R² mentioned above independently may be H, alkyl, alkylhalo or halogen, but they are not limited thereto.

For the foregoing R¹ and R², the alkyl mentioned above may comprise C₁-C₂₄ alkyl, such as C₂-C₂₃ alkyl, C₃-C₂₂ alkyl, C₄-C₂₁ alkyl, C₅-C₂₀ alkyl, C₆-C₁₉ alkyl, C₇-C₁₈ alkyl, C₈-C₁₇ alkyl, C₉-C₁₆ alkyl, C₁₀-C₁₅ alkyl, C₁₁-C₁₄ alkyl, C₁₂-C₁₃ alkyl, etc., but it is not limited thereto. Moreover, in one embodiment, the C₁-C₂₄ alkyl mentioned above may be C₁-C₂₄ straight-chain alkyl or C₄-C₂₄ branched-chain alkyl. Examples of the C₁-C₂₄ straight-chain alkyl mentioned above may comprise, but are not limited to, C₂-C₂₃ straight-chain alkyl, C₃-C₂₂ straight-chain alkyl, C₄-C₂₁ straight-chain alkyl, C₅-C₂₀ straight-chain alkyl, C₆-C₁₉ straight-chain alkyl, C₇-C₁₅ straight-chain alkyl, C₈-C₁₇ straight-chain alkyl, C₉-C₁₆ straight-chain alkyl, C₁₀-C₁₅ straight-chain alkyl, C₁₁-C₁₄ straight-chain alkyl, C₁₂-C₁₃ straight-chain alkyl, etc. Furthermore, examples of the C₄-C₂₄ branched-chain alkyl mentioned above may comprise C₅-C₂₃ branched-chain alkyl, C₆-C₂₂ branched-chain alkyl, C₇-C₂₁ branched-chain alkyl, C₈-C₂₀ branched-chain alkyl, C₉-C₁₉ branched-chain alkyl, C₁₀-C₁₈ branched-chain alkyl, C₁₁-C₁₇ branched-chain alkyl, C₁₂-C₁₆ branched-chain alkyl, C₁₃-C₁₅ branched-chain alkyl etc., but they are not limited thereto.

For the foregoing R¹ and R², the alkylhalo mentioned above may comprise C₁-C₂₄ alkylhalo, such as C₂-C₂₃ alkylhalo, C₃-C₂₂ alkylhalo, C₄-C₂₁ alkylhalo, C₅-C₂₀ alkylhalo, C₆-C₁₉ alkylhalo, C₇-C₁₅ alkylhalo, C₈-C₁₇ alkylhalo, C₉-C₁₆ alkylhalo, C₁₀-C₁₅ alkylhalo, C₁₁-C₁₄ alkylhalo, C₁₂-C₁₃ alkylhalo, but it is not limited thereto. Moreover, in one embodiment, the C₁-C₂₄ alkylhalo mentioned above may be C₁-C₂₄ straight-chain alkylhalo or C₄-C₂₄ branched-chain alkylhalo. Examples of the C₁-C₂₄ straight-chain alkylhalo mentioned above may comprise, but are not limited to, C₂-C₂₃ straight-chain alkylhalo, C₃-C₂₂ straight-chain alkylhalo, C₄-C₂₁ straight-chain alkylhalo, C₅-C₂₀ straight-chain alkylhalo, C₆-C₁₉ straight-chain alkylhalo, C₇-C₁₈ straight-chain alkylhalo, C₅-C₁₇ straight-chain alkylhalo, C₉-C₁₆ straight-chain alkylhalo, C₁₀-C₁₅ straight-chain alkylhalo, C₁-C₁₄ straight-chain alkylhalo, C₁₂-C₁₃ straight-chain alkylhalo, etc. Furthermore, examples of the C₄-C₂₄ branched-chain alkylhalo mentioned above may comprise C₅-C₂₃ branched-chain alkylhalo, C₆-C₂₂ branched-chain alkylhalo, C₇-C₂₁ branched-chain alkylhalo, C₈-C₂₀ branched-chain alkylhalo, C₉-C₁₉ branched-chain alkylhalo, C₁₀-C₁₈ branched-chain alkylhalo, C₁₁-C₁₇ branched-chain alkylhalo, C₁₂-C₁₆ branched-chain alkylhalo, C₁₃-C₁₅ branched-chain alkylhalo etc., but they are not limited thereto.

Furthermore, the alkylhalo mentioned above may be alkyl substituted by at least one halogen, and the at least one halogen mentioned above may be independently selected from F, Cl, Br, I, etc., but it is not limited thereto.

In addition, for the foregoing R¹ and R², the halogen mentioned above may be independently selected from F, Cl, Br, I, etc., but it is not limited thereto.

The foregoing R³ and R⁴ independently may be H or alkyl, but they are not limited thereto. For the foregoing R¹ and R², the alkyl mentioned above may comprise C₁-C₂₄ alkyl, such as C₂-C₂₃ alkyl, C₃-C₂₂ alkyl, C₄-C₂₁ alkyl, C₅-C₂₀ alkyl, C₆-C₁₉ alkyl, C₇-C₁₈ alkyl, C₈-C₁₇ alkyl, C₉-C₁₆ alkyl, C₁₀-C₁₅ alkyl, C₁₁-C₁₄ alkyl, C₁₂-C₁₃ alkyl, etc., but it is not limited thereto. Moreover, in one embodiment, the C₁-C₂₄ alkyl mentioned above may be C₁-C₂₄ straight-chain alkyl or C₄-C₂₄ branched-chain alkyl. Examples of the C₁-C₂₄ straight-chain alkyl mentioned above may comprise, but are not limited to, C₂-C₂₃ straight-chain alkyl, C₃-C₂₂ straight-chain alkyl, C₄-C₂₁ straight-chain alkyl, C₅-C₂₀ straight-chain alkyl, C₆-C₁₉ straight-chain alkyl, C₇-C₁₅ straight-chain alkyl, C₈-C₁₇ straight-chain alkyl, C₉-C₁₆ straight-chain alkyl, C₁₀-C₁₅ straight-chain alkyl, C₁₂-C₁₄ straight-chain alkyl, C₁₂-C₁₃ straight-chain alkyl, etc. Furthermore, examples of the C₄-C₂₄ branched-chain alkyl mentioned above may comprise C₅-C₂₃ branched-chain alkyl, C₆-C₂₂ branched-chain alkyl, C₇-C₂₁ branched-chain alkyl, C₈-C₂₀ branched-chain alkyl, C₉-C₁₉ branched-chain alkyl, C₁₀-C₁₈ branched-chain alkyl, C₁-C₁₇ branched-chain alkyl, C₁₂-C₁₆ branched-chain alkyl, C₁₃-C₁₅ branched-chain alkyl etc., but they are not limited thereto.

Moreover, in Formula (I) shown above, Z may be O or S, but it is not limited thereto.

Furthermore, in Formula (I) shown above, n may be 0 or 1, but it also is not limited thereto.

In addition, in Formula (I) shown above, Ar¹ and Ar² may be independently unsubstituted or substituted aryl or heteroaryl. The foregoing substituted aryl or heteroaryl may be substituted by at least one following substituent, but is not limited thereto: halogen, cyano, NH₂, NHR⁵, NR⁶R⁷, NO₂, alkyl, OH, CF₃, —O-alkyl, —SO₂-alkyl, —SO₂N-alkyl, —S— alkyl, (C₁-C₄)alkoxy(C₁-C₄)alkyl, etc. The foregoing R⁵ and R⁶ may be independently alkyl, and R⁶ and R⁷ are the same, and the alkyl may be unsubstituted or substituted straight-chain alkyl or branched-chain alkyl, and the substituted straight-chain alkyl or branched-chain alkyl may be substituted by at least one following substituent, but is not limited thereto: halogen, oxygen, amino, etc.

In one embodiment, in Formula (I) shown above, Ar¹ and Ar² may be independently unsubstituted or substituted aryl or heteroaryl, and the foregoing substituted aryl or heteroaryl may be substituted by at least one substituent, and the foregoing substituent may comprise, but is not limited to, phenyl, C₁-C₆ alkyl, halogen, alkylhalo, cyano, etc. In this embodiment, Ar¹ and Ar² may be independently selected from, but is not limited to:

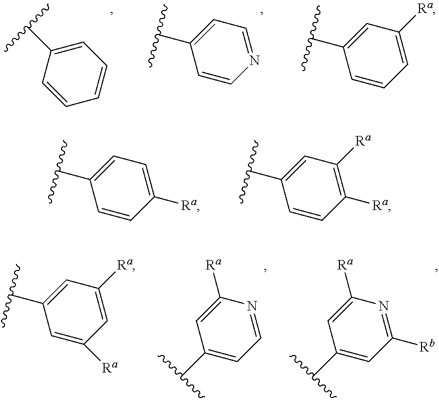

etc. R^{a} and R^{b} may be independently selected from, but is not limited to phenyl, C₁-C₆ alkyl, halogen, alkylhalo, cyano, etc. When R^{a} is halogen, it may be independently selected from F, Cl, Br, I, etc., but it is not limited thereto. When R^{b} is halogen, it may be also independently selected from F, Cl, Br, I, etc., but it is also not limited thereto.

In one specific embodiment, Ar¹ and Ar² may be independently selected from

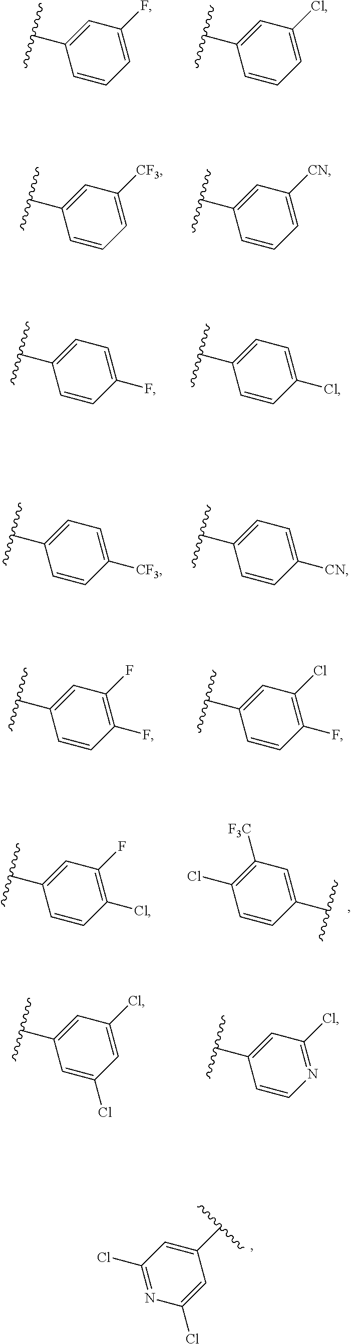

etc., but they are not limited thereto.

In one embodiment, in Formula (I) shown above, Q¹ and X² are both N, Q² is CR², and X¹ is CH. Moreover, in this embodiment, in Formula (I) shown above, R² may be H, alkyl, alkylhalo or halogen.

In one specific embodiment, in Formula (I) shown above, Q¹ and X² are both N, Q² is CR², and X¹ is CH, wherein R² is H. Moreover, in this specific embodiment, Ar¹ may be selected from:

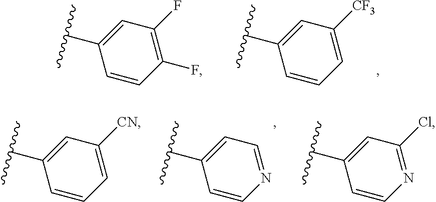

etc., but it is not limited thereto. Ar² may be selected from, but is not limited to:

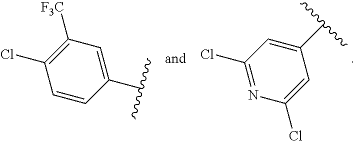 [SMILES: *c1ccc(Cl)c(C(F)(F)F)c1 ; *c1cc(Cl)nc(Cl)c1]

In addition, in another specific embodiment, in Formula (I) shown above, Q¹ and X² are both N, Q² is CR², and X¹ is CH, wherein R² is halogen. Moreover, in this specific embodiment, Ar¹ may be

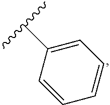 [SMILES: *c1ccccc1]

etc., but it is not limited thereto. Ar² may be

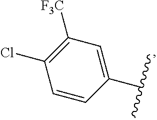 [SMILES: *c1ccc(Cl)c(C(F)(F)F)c1]

etc., but it is also not limited thereto.

In another embodiment, in Formula (I) shown above, Q¹ and Q² are both N, and X¹ and X² are both CH. In this embodiment, Ar¹ may be

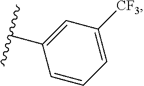 [SMILES: *c1cccc(C(F)(F)F)c1]

but it is not limited thereto while Ar² may be

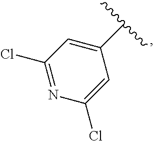 [SMILES: *c1cc(Cl)nc(Cl)c1]

but it is also not limited thereto.

In further another embodiment, in Formula (I) shown above, X² is N, Q¹ is CR¹, Q² is CR², and X¹ is CH. Moreover, in this embodiment, in Formula (I) shown above, R¹ and R² independently may be H, alkyl, alkylhalo or halogen, but they are not limited thereto.

In one specific embodiment, in Formula (I) shown above, X² is N, Q¹ is CR¹, Q² is CR², and X¹ is CH, wherein R¹ and R² both may be H. Moreover, in this specific embodiment, Ar¹ may be selected from, but is not limited to,

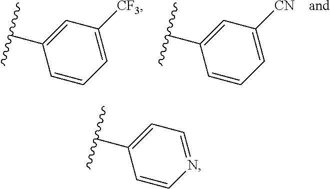

etc. while Ar² may be selected from:

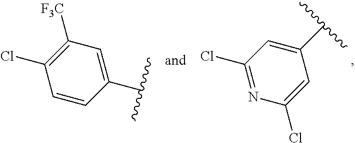 [SMILES: *c1ccc(Cl)c(C(F)(F)F)c1 ; *c1cc(Cl)nc(Cl)c1]

etc., but it is also not limited thereto.

In one embodiment, the small molecule compound of the present disclosure may comprise, but is not limited to,

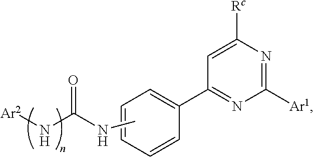

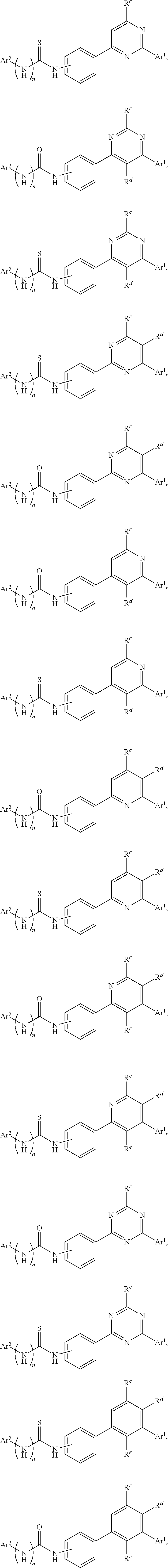

etc. R^{c}, R^{d} and R^{e} are independently H or alkyl, etc., but they are not limited thereto.

In addition, in one embodiment, the small molecule compound of the present disclosure may comprise one of the following Compound 1 to Compound 15, but is not limited thereto:

| Compound | |
|---|---|
| Number | Structure |
| 1 | 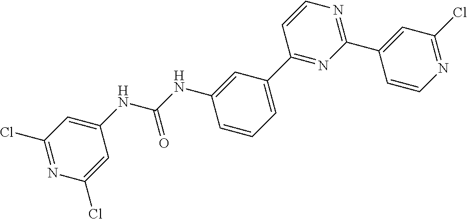 [SMILES: O=C(Nc1cccc(-c2ccnc(-c3ccnc(Cl)c3)n2)c1)Nc1cc(Cl)nc(Cl)c1] |
| 2 | 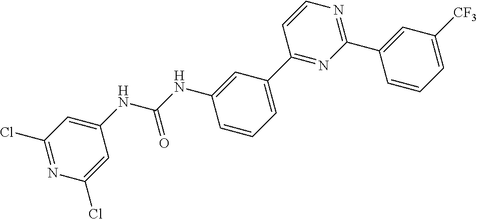 [SMILES: O=C(Nc1cccc(-c2ccnc(-c3cccc(C(F)(F)F)c3)n2)c1)Nc1cc(Cl)nc(Cl)c1] |
| 3 | 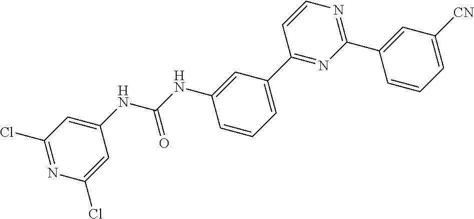 [SMILES: N#Cc1cccc(-c2nccc(-c3cccc(NC(=O)Nc4cc(Cl)nc(Cl)c4)c3)n2)c1] |
| 4 | 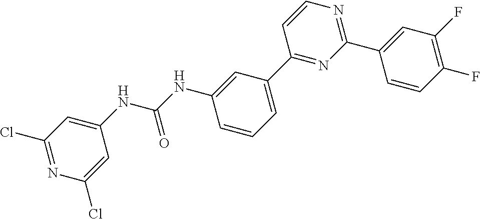 [SMILES: O=C(Nc1cccc(-c2ccnc(-c3ccc(F)c(F)c3)n2)c1)Nc1cc(Cl)nc(Cl)c1] |
| 5 | 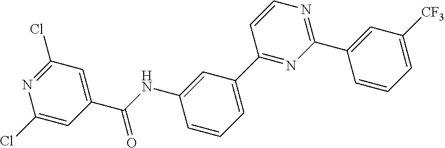 [SMILES: O=C(Nc1cccc(-c2ccnc(-c3cccc(C(F)(F)F)c3)n2)c1)c1cc(Cl)nc(Cl)c1] |
| 6 | 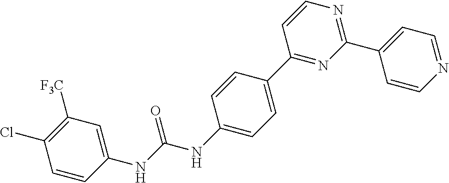 [SMILES: O=C(Nc1ccc(-c2ccnc(-c3ccncc3)n2)cc1)Nc1ccc(Cl)c(C(F)(F)F)c1] |
| 7 | 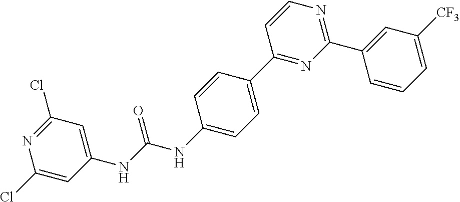 [SMILES: O=C(Nc1ccc(-c2ccnc(-c3cccc(C(F)(F)F)c3)n2)cc1)Nc1cc(Cl)nc(Cl)c1] |
| 8 | 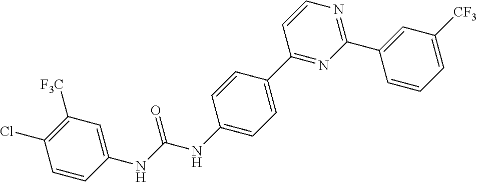 [SMILES: O=C(Nc1ccc(-c2ccnc(-c3cccc(C(F)(F)F)c3)n2)cc1)Nc1ccc(Cl)c(C(F)(F)F)c1] |
| 9 | 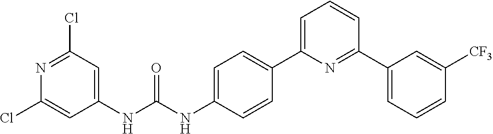 [SMILES: O=C(Nc1ccc(-c2cccc(-c3cccc(C(F)(F)F)c3)n2)cc1)Nc1cc(Cl)nc(Cl)c1] |
| 10 | 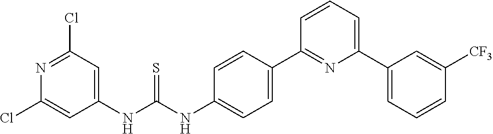 [SMILES: FC(F)(F)c1cccc(-c2cccc(-c3ccc(NC(=S)Nc4cc(Cl)nc(Cl)c4)cc3)n2)c1] |
| 11 | 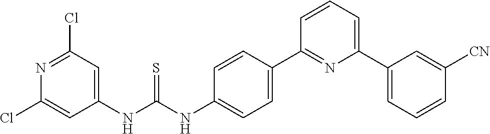 [SMILES: N#Cc1cccc(-c2cccc(-c3ccc(NC(=S)Nc4cc(Cl)nc(Cl)c4)cc3)n2)c1] |
| 12 | 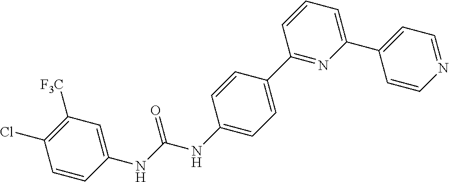 [SMILES: O=C(Nc1ccc(-c2cccc(-c3ccncc3)n2)cc1)Nc1ccc(Cl)c(C(F)(F)F)c1] |
| 13 | 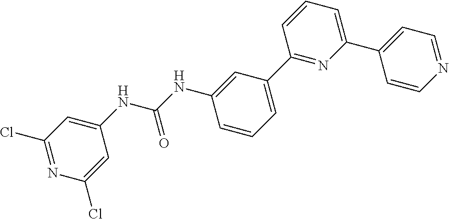 [SMILES: O=C(Nc1cccc(-c2cccc(-c3ccncc3)n2)c1)Nc1cc(Cl)nc(Cl)c1] |
| 14 | 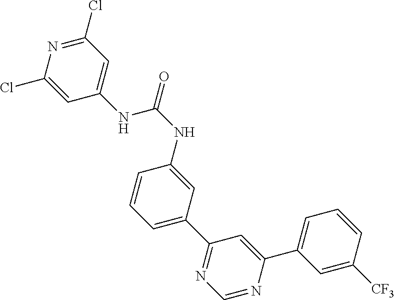 [SMILES: O=C(Nc1cccc(-c2cc(-c3cccc(C(F)(F)F)c3)ncn2)c1)Nc1cc(Cl)nc(Cl)c1] |
| 15 | 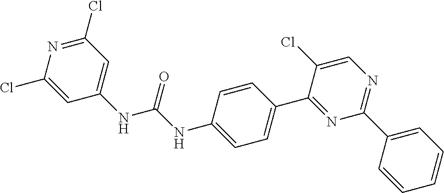 [SMILES: O=C(Nc1ccc(-c2nc(-c3ccccc3)ncc2Cl)cc1)Nc1cc(Cl)nc(Cl)c1] |

In one specific embodiment, the small molecule compound of the present disclosure may be any one of Compound 1 to Compound 15 mentioned above, but is not limited thereto.

Since as mentioned above, the small molecule compound or salt thereof or solvates of them of the present disclosure may have an effect of inhibiting GNAQ, the present disclosure may also provide a GNAQ inhibitor which may comprise, but is not limited to any small molecule compound or salt thereof or solvates of them of the present disclosure mentioned above.

In one embodiment, in the GNAQ inhibitor of the present disclosure mentioned above, the small molecule compound of the present disclosure mentioned above may comprise any one of Compound 1 to Compound 15 mentioned above, but it is not limited thereto.

Moreover, according to the foregoing, the present disclosure may also provide a use of any aforementioned small molecule compound or salt thereof or solvates of them of the present disclosure as a GNAQ inhibitor. Furthermore, the present disclosure may also provide a use of any aforementioned small molecule compound or salt thereof or solvates of them of the present disclosure in the manufacture of a GNAQ inhibitor.

In one embodiment, in the foregoing use of any aforementioned small molecule compound or salt thereof or solvates of them of the present disclosure as a GNAQ inhibitor, or in the foregoing use of any aforementioned small molecule compound or salt thereof or solvates of them of the present disclosure in the manufacture of a GNAQ inhibitor, the small molecule compound of the present disclosure mentioned above may comprise, but is not limited to, any one of Compound 1 to Compound 15 mentioned above.

In addition, since as mentioned above, the small molecule compound or salt thereof or solvates of them of the present disclosure may have an effect of treating and/or preventing a disease, the present disclosure may further provide a pharmaceutical composition which may comprises any aforementioned small molecule compound or salt thereof or solvates of them of the present disclosure, but it is not limited thereto.

In one embodiment, in the foregoing pharmaceutical composition of the present disclosure, the small molecule compound of the present disclosure mentioned above may comprise any one of Compound 1 to Compound 15 mentioned above, but it is not limited thereto.

Furthermore, in one embodiment, the foregoing pharmaceutical composition of the present disclosure may further comprise a pharmaceutically acceptable carrier or salt, but it is not limited thereto.

The pharmaceutically acceptable carrier mentioned above may comprise, but is not limited to, a solvent, a dispersion medium, a coating, an antibacterial and antifungal agent, or an isotonic and absorption delaying agent, etc. which is suitable for pharmaceutical administration. The pharmaceutical composition can be formulated into dosage forms for different administration routes utilizing conventional methods.

Moreover, the pharmaceutically acceptable salt mentioned above may comprise, but is not limited to, salts including inorganic cation, such as alkali metal salts such as sodium salt, potassium salt or amine salt, such as alkaline-earth metal salt such as magnesium salt or calcium salt, such as the salt containing bivalent or quadrivalent cation such as zinc salt, aluminum salt or zirconium salt. In addition, the pharmaceutically acceptable salt may also be organic salt, such as dicyclohexylamine salt, methyl-D-glucamine, and amino acid salt such as arginine, lysine, histidine, or glutamine.

Furthermore, the pharmaceutical composition of the present disclosure may be administered to a subject in need of this pharmaceutical composition, but it is not limited thereto. Moreover, the routes of administration of the pharmaceutical compositions of the present disclosure may include parenteral, oral, by an inhalation spray, or via an implanted reservoir, but it is not limited thereto. The parenteral manner may comprise, but is not limited to, smearing on affected area, subcutaneous, intracutaneous, intravenous, intramuscular, intra-articular, intra-arterial, intrasynovial, intrasternal, intrathecal, intralesional injection, ophthalmic external use, intraocular injection as well as infusion techniques, etc.

Forms of topical application for smearing may include an ointment, an emulsion, a liquid, a gel, etc., but they are not limited thereto. In addition, forms of application for ophthalmic external use may include, but are not limited to, an eye drop, an eye ointment, an eye gel, etc.

Furthermore, the foregoing subject need to be administered the pharmaceutical composition may include, but is not limited to, a vertebrate. Moreover, the vertebrate mentioned above may include a fish, an amphibian, a reptile, a bird, or a mammal, but it is not limited thereto. Examples of the mammal may include, but are not limited to, a human, an orangutan, a monkey, a horse, a donkey, a goat, a sheep, a dog, a cat, a rabbit, a guinea pig, a rat, and a mouse. In one embodiment, the subject mentioned above may be a human.

In one embodiment, the pharmaceutical composition of the present disclosure mentioned above may be used to treat and/or prevent a GNAQ-related disease and/or symptom, but it is not limited thereto.

There are no specific limitations to the GNAQ-related disease and/or symptom described herein, as long as the mechanism and/or progression of a disease and/or symptom involves the expression and/or mutation of GNAQ or is affected and/or influenced by GNAQ.

The GNAQ-related disease and/or symptom may comprise, but is not limited to a cancer, Sturge-Weber syndrome, Klippel-Trenaunay syndrome, port wine stain, etc. Examples of the cancer described herein may include, but are not limited to, melanoma, lymphoma, liver cancer, breast cancer, etc. The above-mentioned melanoma may include uveal melanoma, but it is not limited thereto. The above-mentioned lymphoma may include, but is not limited to, NK/T cell lymphoma. Moreover, the breast cancer mentioned above may include mammary gland cancer, triple-negative breast cancer, etc., but it is not limited thereto. In one specific embodiment, the GNAQ-related disease and/or symptom described herein is breast cancer.

Furthermore, the pharmaceutical composition of the present disclosure may be formulated into a pharmaceutical preparation, but it is not limited thereto. The pharmaceutical composition of the present disclosure may be formulated into an oral preparation or a parenteral preparation. The above-mentioned oral preparation may include, but is not limited to, tablets, granules, powders, pellets, capsules, coated tablets, emulsions, solutions, aqueous suspensions, and dispersions, instant powders, etc., but it is not limited thereto. The above-mentioned parenteral preparation may include injections, ophthalmic/ear/nasal emulsions, ophthalmic/ear/nasal solutions, semi-solid preparations (such as ointments, gels), etc., but it is not limited thereto.

In addition, the present disclosure may also provide a use of any aforementioned small molecule compound or salt thereof or solvates of them of the present disclosure in the manufacture of a medicament.

In one embodiment, the medicament mentioned above may be used to treat and/or prevent a GNAQ-related disease and/or symptom, but it is not limited thereto. The relevant descriptions of the GNAQ-related disease and/or symptom are as mentioned above, and thus they will not be repeated here. In one specific embodiment, the GNAQ-related disease and/or symptom described herein is breast cancer.

Moreover, in one embodiment, in the foregoing use of any aforementioned small molecule compound or salt thereof or solvates of them of the present disclosure in the manufacture of a medicament, the small molecule compound of the present disclosure mentioned above may comprise, but is not limited to, any one of Compound 1 to Compound 15 mentioned above.

In one embodiment, in the foregoing use of any aforementioned small molecule compound or salt thereof or solvates of them of the present disclosure in the manufacture of a medicament, in another embodiment, a pharmaceutically acceptable carrier or salt may be used together with any aforementioned small molecule compound or salt thereof or solvates of them of the present disclosure to prepare the above-mentioned medicament. The relevant descriptions of the pharmaceutically acceptable carrier or salt are as mentioned above, and thus they will not be repeated here.

Based on the foregoing, the present disclosure may further provide a pharmaceutical composition for treating and/or preventing a GNAQ-related disease and/or symptom, which may comprise any aforementioned small molecule compound or salt thereof or solvates of them of the present disclosure, but it is not limited thereto. Similarly, the relevant descriptions of the GNAQ-related disease and/or symptom are as mentioned above, and thus they will not be repeated here. In one embodiment, the GNAQ-related disease and/or symptom described herein is a cancer. In one specific embodiment, the GNAQ-related disease and/or symptom described herein is breast cancer.

In one embodiment, in the foregoing pharmaceutical composition for treating and/or preventing a GNAQ-related disease and/or symptom of the present disclosure, the small molecule compound of the present disclosure mentioned above may comprise, but is not limited to, any one of Compound 1 to Compound 15 mentioned above.

Furthermore, in one embodiment, the foregoing pharmaceutical composition for treating and/or preventing a GNAQ-related disease and/or symptom of the present disclosure may further comprise a pharmaceutically acceptable carrier or salt, but it is not limited thereto. Regarding the said pharmaceutically acceptable carrier or salt, the relevant descriptions for the pharmaceutically acceptable carrier or salt in the above contents related to the pharmaceutical composition of the present disclosure can be referred to, and thus they will not be repeated here.

The foregoing pharmaceutical composition for treating and/or preventing a GNAQ-related disease and/or symptom of the present disclosure may be administered to a subject in need thereof, but it is not limited thereto. Regarding the administration route of the pharmaceutical composition for treating and/or preventing a GNAQ-related disease and/or symptom of the present disclosure and the subject in need thereof, the relevant descriptions thereof can be referred to the relevant descriptions for the administration route and the subject in the above contents related to the pharmaceutical composition of the present disclosure, and thus they will not be repeated here.

The foregoing pharmaceutical composition for treating and/or preventing a GNAQ-related disease and/or symptom of the present disclosure may also be formulated into a pharmaceutical preparation, but it is not limited thereto. Regarding types of pharmaceutical preparations that can be prepared from the pharmaceutical composition for treating and/or preventing a GNAQ-related disease and/or symptom of the present disclosure, the relevant descriptions thereof can be referred to the relevant descriptions for the pharmaceutical preparation in the above contents related to the pharmaceutical composition of the present disclosure, and thus they will not be repeated here.

In addition, based on the foregoing, the present disclosure may also provide a use of any aforementioned small molecule compound or salt thereof or solvates of them of the present disclosure in the manufacture of a medicament for treating and/or preventing a GNAQ-related disease and/or symptom.

The relevant descriptions of the GNAQ-related disease and/or symptom are as mentioned above, and thus they will not be repeated here.

Furthermore, in one embodiment, in the foregoing use of any aforementioned small molecule compound or salt thereof or solvates of them of the present disclosure in the manufacture of a medicament for treating and/or preventing a GNAQ-related disease and/or symptom, the small molecule compound of the present disclosure mentioned above may comprise, but is not limited to, any one of Compound 1 to Compound 15 mentioned above.

In the foregoing use of any aforementioned small molecule compound or salt thereof or solvates of them of the present disclosure in the manufacture of a medicament for treating and/or preventing a GNAQ-related disease and/or symptom, in another embodiment, a pharmaceutically acceptable carrier or salt may be used together with any aforementioned small molecule compound or salt thereof or solvates of them of the present disclosure to prepare the above-mentioned medicament. The relevant descriptions of the pharmaceutically acceptable carrier or salt are as mentioned above, and thus they will not be repeated here.

In addition, based on the foregoing, the present disclosure may also provide a method for treating and/or preventing a GNAQ-related disease and/or symptom. The above-mentioned method for treating and/or preventing a GNAQ-related disease and/or symptom of the present disclosure may comprise, but is not limited to, administering any aforementioned small molecule compound or salt thereof or solvates of them of the present disclosure, or any aforementioned pharmaceutical composition for treating and/or preventing a GNAQ-related disease and/or symptom of the present disclosure, to a subject in need thereof. Regarding the subject in need of any aforementioned small molecule compound or salt thereof or solvates of them of the present disclosure, or any aforementioned pharmaceutical composition for treating and/or preventing a GNAQ-related disease and/or symptom of the present disclosure, the relevant descriptions thereof can be referred to the relevant descriptions for the subject in the above contents related to the pharmaceutical composition of the present disclosure, and thus they will not be repeated here.

Furthermore, also based on the foregoing, the present disclosure further provides a pharmaceutical composition for treating and/or preventing a cancer, which may comprise any aforementioned small molecule compound or salt thereof or solvates of them of the present disclosure, but it is not limited thereto. The cancer described herein may include, but are not limited to, melanoma, lymphoma, liver cancer, breast cancer, etc. The above-mentioned melanoma may include uveal melanoma, but it is not limited thereto. The above-mentioned lymphoma may include, but is not limited to, NK/T cell lymphoma. Moreover, the breast cancer mentioned above may include mammary gland cancer, triple-negative breast cancer, etc., but it is not limited thereto. In one specific embodiment, the cancer described herein is breast cancer.

In one embodiment, in the foregoing pharmaceutical composition for treating and/or preventing a cancer of the present disclosure, the small molecule compound of the present disclosure mentioned above may comprise, but is not limited to, any one of Compound 1 to Compound 15 mentioned above.

Furthermore, in one embodiment, the foregoing pharmaceutical composition for treating and/or preventing a cancer of the present disclosure may further comprise a pharmaceutically acceptable carrier or salt, but it is not limited thereto. Regarding the said pharmaceutically acceptable carrier or salt, the above relevant descriptions for the pharmaceutically acceptable carrier or salt in the above contents related to the pharmaceutical composition of the present disclosure can be referred to, and thus they will not be repeated here.

The foregoing pharmaceutical composition for treating and/or preventing a cancer of the present disclosure may be administered to a subject in need thereof, but it is not limited thereto. Regarding the administration route of the pharmaceutical composition for treating and/or preventing a cancer of the present disclosure and the subject in need thereof, the relevant descriptions thereof can be referred to the relevant descriptions for the administration route and the subject in the above contents related to the pharmaceutical composition of the present disclosure, and thus they will not be repeated here.

The foregoing pharmaceutical composition for treating and/or preventing a cancer of the present disclosure may also be formulated into a pharmaceutical preparation, but it is not limited thereto. Regarding types of pharmaceutical preparations that can be prepared from the pharmaceutical composition for treating and/or preventing a cancer of the present disclosure, the relevant descriptions thereof can be referred to the relevant descriptions for the pharmaceutical preparation in the above contents related to the pharmaceutical composition of the present disclosure, and thus they will also not be repeated here.

In addition, based on the foregoing, the present disclosure may also provide a use of any aforementioned small molecule compound or salt thereof or solvates of them of the present disclosure in the manufacture of a medicament for treating and/or preventing a cancer.

The relevant descriptions of the cancer described herein are as mentioned above, and thus they will not be repeated here. In one specific embodiment, the cancer described herein is breast cancer.

Furthermore, in one embodiment, in the foregoing use of any aforementioned small molecule compound or salt thereof or solvates of them of the present disclosure in the manufacture of a medicament for treating and/or preventing a cancer, the small molecule compound of the present disclosure mentioned above may comprise, but is not limited to, any one of Compound 1 to Compound 15 mentioned above.

In the foregoing use of any aforementioned small molecule compound or salt thereof or solvates of them of the present disclosure in the manufacture of a medicament for treating and/or preventing a cancer, in another embodiment, a pharmaceutically acceptable carrier or salt may be used together with any aforementioned small molecule compound or salt thereof or solvates of them of the present disclosure to prepare the above-mentioned medicament. The relevant descriptions of the pharmaceutically acceptable carrier or salt are as mentioned above, and thus they will not be repeated here.

Moreover, based on the foregoing, the present disclosure may also provide a method for treating and/or preventing a cancer. The above-mentioned method for treating and/or preventing a cancer of the present disclosure may comprise, but is not limited to, administering any aforementioned small molecule compound or salt thereof or solvates of them of the present disclosure, or any aforementioned pharmaceutical composition for treating and/or preventing a cancer of the present disclosure, to a subject in need thereof.

The relevant descriptions of the cancer described herein are as mentioned above, and thus they will not be repeated here. In one specific embodiment, the cancer described herein is breast cancer.

Regarding the subject in need of any aforementioned small molecule compound or salt thereof or solvates of them of the present disclosure or any aforementioned pharmaceutical composition for treating and/or preventing a cancer of the present disclosure, the relevant descriptions thereof can be referred to the relevant descriptions for the subject in the above contents related to the pharmaceutical composition of the present disclosure, and thus they will not be repeated here.

### Examples

A. Compound abbreviation
ACT: Acetone
EA: Ethyl acetate
DCM: Dichloromethane
T3P: Propanephosphonic acid anhydride (PPAA)
B. Preparation of compounds
Preparation of Compound 1 to Compound 15
1. Preparation of Compound 1

The synthesis scheme of Compound 1 is shown in Scheme 1 below:

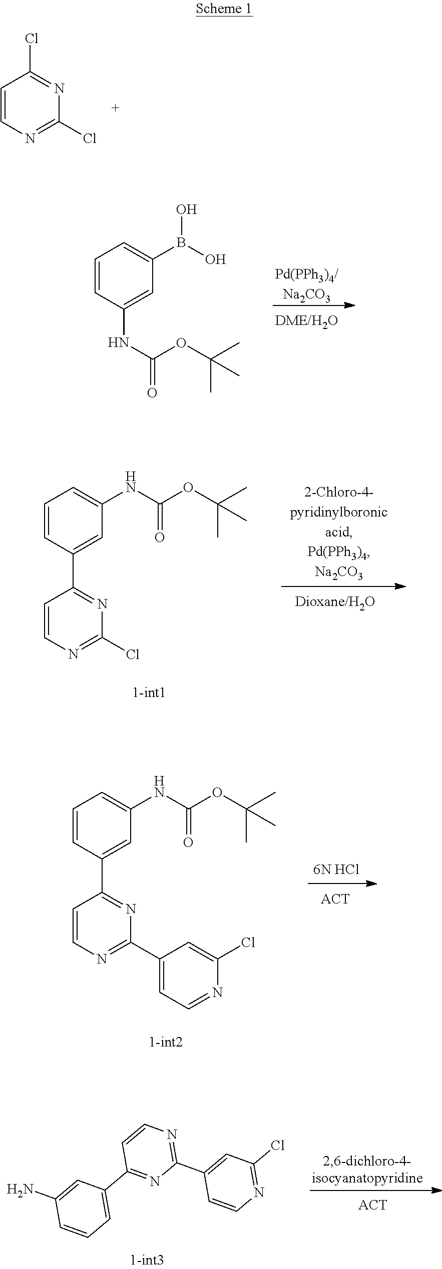

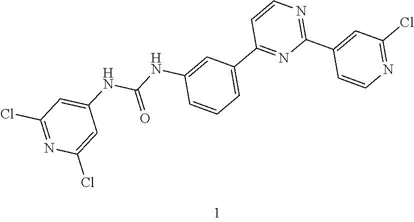 [SMILES: O=C(Nc1cccc(-c2ccnc(-c3ccnc(Cl)c3)n2)c1)Nc1cc(Cl)nc(Cl)c1]

### (1) Preparation of Intermediate 1-Int1

149 mg (1.0 eq) of 2,4-dichloropyrimidine was placed in a reaction flask under N₂, and 1.0 eq of 3-(N-Boc-amino)phenylboronic acid, 2.5 eq of Na₂CO₃ saturated aqueous solution and 2 mL of 1,2-dimethoxyethane were added to the reaction flask, separately. Next, after 4.5% mole Pd(PPh₃)₄ was added to the reaction flask, the solution in the reaction flask was refluxed and stirred for 2 hours. After the completion of the reaction was confirmed by thin layer chromatography (TLC) (hexane/EA=8:1), 1,2-dimethoxyethane was removed from the solution in the reaction flask under reduced pressure. After that, the solution in reaction flask was added to water and extracted with EA. After the extraction, the EA layer was taken out. The EA layer was dried, concentrated, and purified with a column to obtain 135 mg of 1-int1 (44%).

### (2) Preparation of Intermediate 1-Int2

135 mg (1.0 eq) of 1-int1 was placed in a reaction flask under N₂, and 1.0 eq of 2-chloro-4-pyridinylboronic acid, 2.5 eq of Na₂CO₃ saturated aqueous solution and 2 mL of 1,4-dioxane were added to the reaction flask, separately. Next, after 4.5% mole Pd(PPh₃)₄ was added to the reaction flask, the solution in the reaction flask was refluxed and stirred for 2 hours. After the completion of the reaction was confirmed by thin layer chromatography (TLC) (hexane/EA=5:1), 1,4-dioxane was removed from the solution in the reaction flask under reduced pressure. After that, the solution in reaction flask was added to water and extracted with EA. After the extraction, the EA layer was taken out. The EA layer was dried, concentrated, and purified with a column to obtain 80 mg of 1-int2 (47%).

### (3) Preparation of Intermediate 1-Int3

80 mg of 1-int2 was completely dissolved in 1 mL of ACT to form a solution, and 6N HCl was added to the solution to react for 3 hours. After the reaction was completed, the liquid of the reacted reactant was sucked to dry, and then saturated NaHCO₃ and dichloromethane (DCM) were added for performing extraction. After the extraction, the organic layer was taken out. The organic layer was dried and concentrated to obtain 56 mg of 1-int3 (95%).

### (4) Preparation of Compound 1

12 mg of 1-int3 was dissolved in 1 mL of ACT to form a solution, and 1 eq of 2,6-dichloro-4-isocyanatopyridine was added to the solution. Next, the solution was stirred at room temperature overnight. After the reaction was completed, the resulting solution was filtered to obtain a solid. The obtained solid was oven-dried to obtain 8 mg of Compound 1 (Yield 42%).

¹H-NMR (400 MHz, DMSO-d₆): δ 9.43 (s, 1H), 9.28 (s, 1H), 9.07 (s, 1H), 8.66 (s, 1H), 8.45 (s, 1H), 8.39 (t, J=10 Hz, 1H), 8.16-8.11 (m, 2H), 7.97 (s, 1H), 7.76 (d, J=6.8 Hz, 1H), 7.68-7.63 (m, 2H), 7.61-7.51 (m, 1H)

### 2. Preparation of Compound 2

The synthesis scheme of Compound 2 is shown in Scheme 2 below:

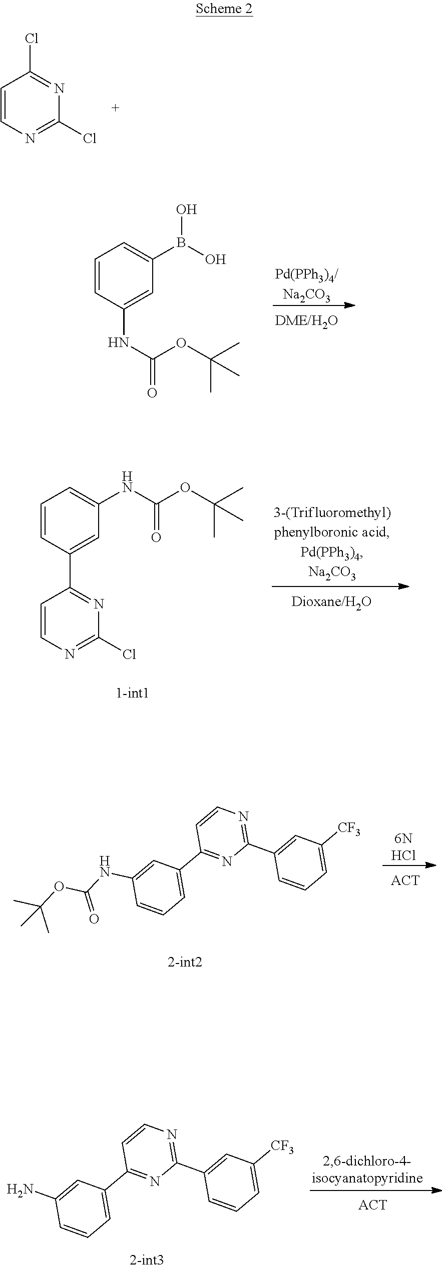

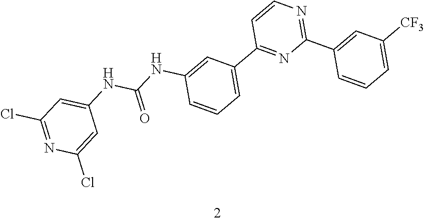 [SMILES: O=C(Nc1cccc(-c2ccnc(-c3cccc(C(F)(F)F)c3)n2)c1)Nc1cc(Cl)nc(Cl)c1]

(1) Preparation of Intermediate 1-int1

1.49 g (1.0 eq) of 2,4-dichloropyrimidine was placed in a reaction flask under N₂, and 1.0 eq of 3-(N-Boc-amino)phenylboronic acid, 2.5 eq of Na₂CO₃ saturated aqueous solution and 20 mL of 1,2-dimethoxyethane were added to the reaction flask, separately. Next, after 4.5% mole Pd(PPh₃)₄ was added to the reaction flask, the solution in the reaction flask was refluxed and stirred for 2 hours. After the completion of the reaction was confirmed by thin layer chromatography (TLC) (hexane/EA=8:1), 1,2-dimethoxyethane was removed from the solution in the reaction flask under reduced pressure. After that, the solution in reaction flask was added to water and extracted with EA. After the extraction, the EA layer was taken out. The EA layer was dried, concentrated, and purified with a column to obtain 2.3 g of 1-int1 (76%).

(2) Preparation of Intermediate 2-int2

0.74 g (1.0 eq) of 1-int1 was placed in a reaction flask under N₂, and 1.0 eq of 3-(trifluoromethyl)phenylboronic acid, 2.5 eq of Na₂CO₃ saturated aqueous solution and 20 mL 1,4-dioxane were added to the reaction flask, separately. Next, after 4.5% mole Pd(PPh₃)₄ was added to the reaction flask, the solution in the reaction flask was refluxed and stirred for 2 hours. After the completion of the reaction was confirmed by thin layer chromatography (TLC) (hexane/EA=5:1), 1,4-dioxane was removed from the solution in the reaction flask under reduced pressure. After that, the solution in reaction flask was added to water and extracted with EA. After the extraction, the EA layer was taken out. The EA layer was dried, concentrated, and purified with a column to obtain 684 mg of 2-int2 (69%).

(3) Preparation of Intermediate 2-int3

550 mg of 2-int2 was completely dissolved in 10 mL of ACT to form a solution, and 6N HCl was added to the solution to react for 3 hours. After the reaction was completed, the liquid of the reacted reactant was sucked to dry, and then saturated NaHCO₃ and DCM were added for performing extraction. After the extraction, the organic layer was taken out. The organic layer was dried and concentrated to obtain 315 mg of 2-int3 (95%).

### (4) Preparation of Compound 2

18 mg of 2-int3 was dissolved in 1 mL of ACT to form a solution, and 1 eq of 2,6-dichloro-4-isocyanatopyridine was added to the solution. Next, the solution was stirred at room temperature overnight. After the reaction was completed, the resulting solution was filtered to obtain a solid. The obtained solid was oven-dried to obtain 9.5 mg of Compound 2 (Yield 34%).

¹H-NMR (500 MHz, DMSO-d₆): δ 9.02 (s, 1H), 8.81 (s, 2H), 8.59 (s, 1H), 8.04 (s, 1H), 8.03-7.96 (m, 1H), 7.83 (t, J=8 Hz, 1H), 7.69 (d, J=7 Hz, 1H), 7.61 (s, 2H), 7.54 (t, J=7.5 Hz, 1H)

### 3. Preparation of Compound 3

The synthesis scheme of Compound 3 is shown in Scheme 3 below:

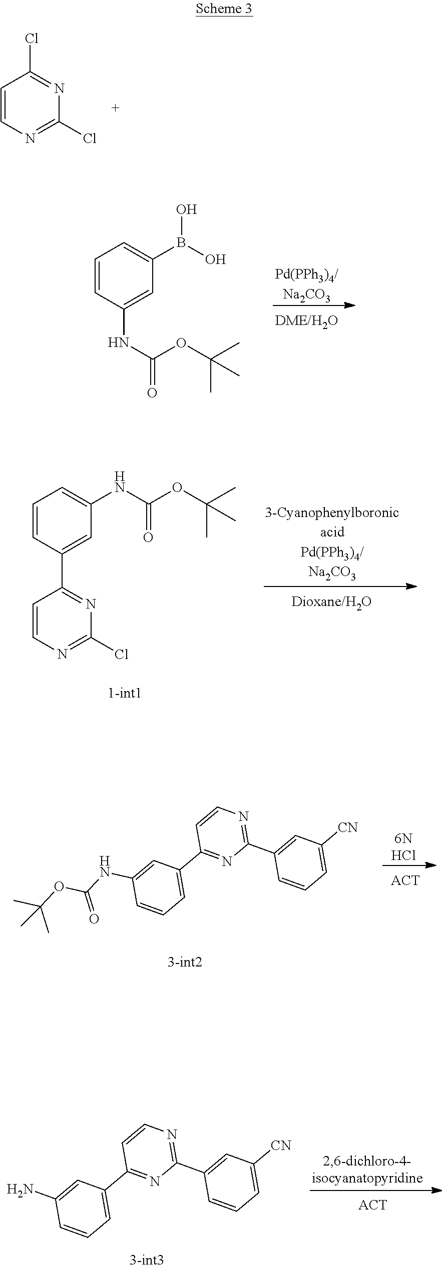

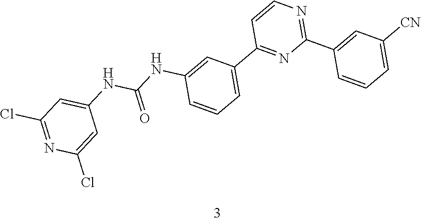 [SMILES: N#Cc1cccc(-c2nccc(-c3cccc(NC(=O)Nc4cc(Cl)nc(Cl)c4)c3)n2)c1]

(1) Preparation of Intermediate 1-int1

The preparation method is the same as the preparation method for Intermediate 1-int1 in the preparation of Compound 2.

(2) Preparation of Intermediate 3-int2

61 mg (1.0 eq) of 1-int1 was placed in a reaction flask under N₂, and 1.0 eq of 3-cyanophenylboronic acid, 2.5 eq of Na₂CO₃ saturated aqueous solution and 5 mL 1,4-dioxane were added to the reaction flask, separately. Next, after 4.5% mole Pd(PPh₃)₄ was added to the reaction flask, the solution in the reaction flask was refluxed and stirred for 2 hours. After the completion of the reaction was confirmed by thin layer chromatography (TLC) (hexane/EA=5:1), 1,4-dioxane was removed from the solution in the reaction flask under reduced pressure. After that, the solution in reaction flask was added to water and extracted with EA. After the extraction, the EA layer was taken out. The EA layer was dried, concentrated, and purified with a column to obtain 55 mg of 3-int2 (60%).

(3) Preparation of Intermediate 3-int3

55 mg of 3-int2 was completely dissolved in 10 mL of ACT to form a solution, and 6N HCl was added to the solution to react for 3 hours. After the reaction was completed, the liquid of the reacted reactant was sucked to dry, and then saturated NaHCO₃ and DCM were added for performing extraction. After the extraction, the organic layer was taken out. The organic layer was dried and concentrated to obtain 30 mg of 3-int3 (75%).

### (4) Preparation of Compound 3

15 mg of 3-int3 was dissolved in 1 mL of ACT to form a solution, and 1 eq of 2,6-dichloro-4-isocyanatopyridine was added to the solution. Next, the solution was stirred at room temperature overnight. After the reaction was completed, the resulting solution was filtered to obtain a solid. The obtained solid was oven-dried to obtain 15 mg of Compound 3 (Yield 59%).

¹H-NMR (500 MHz, DMSO-d⁶): δ 9.69 (s, 1H), 9.43 (s, 1H), 9.02 (d, J=5 Hz, 1H), 8.84 (s, 1H), 8.81 (d, J=7.5 Hz, 1H), 8.43 (s, 1H), 8.06-8.01 (m, 3H), 7.82-7.76 (m, 2H), 7.59 (s, 2H), 7.56-7.54 (m, 1H)

### 4. Preparation of Compound 4

The synthesis scheme of Compound 4 is shown in Scheme 4 below:

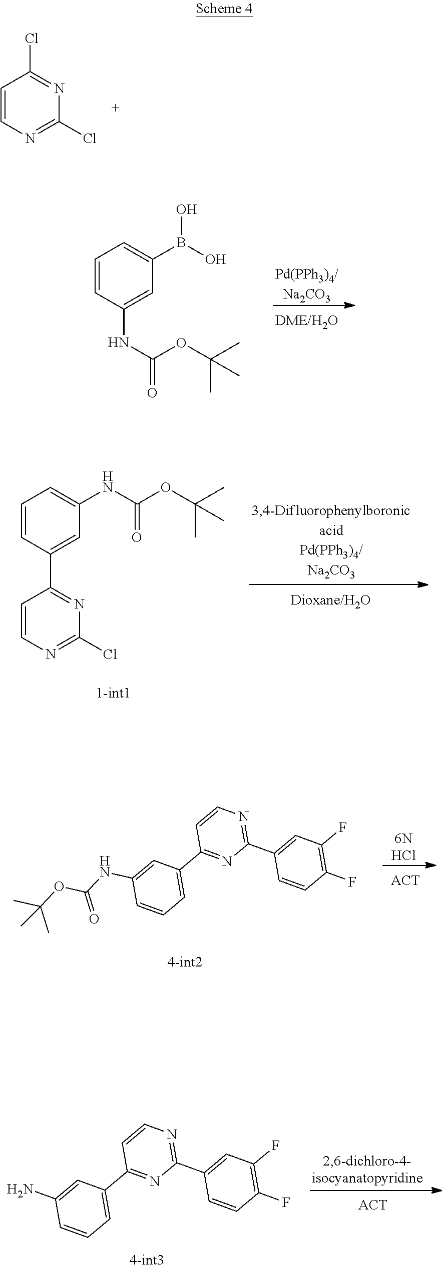

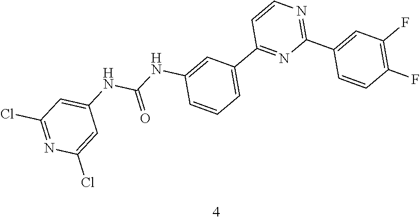 [SMILES: O=C(Nc1cccc(-c2ccnc(-c3ccc(F)c(F)c3)n2)c1)Nc1cc(Cl)nc(Cl)c1]

(1) Preparation of Intermediate 1-int1

The preparation method is the same as the preparation method for Intermediate 1-int1 in the preparation of Compound 2.

(2) Preparation of Intermediate 4-int2

80 mg (1.0 eq) of 1-int1 was placed in a reaction flask under N₂, and 1.0 eq of 3,4-difluorophenylboronic acid, 2.5 eq of Na₂CO₃ saturated aqueous solution and 5 mL 1,4-dioxane were added to the reaction flask, separately. Next, after 4.5% mole Pd(PPh₃)₄ was added to the reaction flask, the solution in the reaction flask was refluxed and stirred for 2 hours. After the completion of the reaction was confirmed by thin layer chromatography (TLC) (hexane/EA=5:1), 1,4-dioxane was removed from the solution in the reaction flask under reduced pressure. After that, the solution in reaction flask was added to water and extracted with EA. After the extraction, the EA layer was taken out. The EA layer was dried, concentrated, and purified with a column to obtain 83 mg of 4-int2 (82%).

(3) Preparation of Intermediate 4-int3

82 mg of 4-int2 was completely dissolved in 10 mL of ACT to form a solution, and 6N HCl was added to the solution to react for 3 hours. After the reaction was completed, the liquid of the reacted reactant was sucked to dry, and then saturated NaHCO₃ and DCM were added for performing extraction. After the extraction, the organic layer was taken out. The organic layer was dried and concentrated to obtain 53 mg of 4-int3 (86%).

### (4) Preparation of Compound 4

28 mg of 4-int3 was dissolved in 1 mL of ACT to form a solution, and 1 eq of 2,6-dichloro-4-isocyanatopyridine was added to the solution. Next, the solution was stirred at room temperature overnight. After the reaction was completed, the resulting solution was filtered to obtain a solid. The obtained solid was oven-dried to obtain 20 mg of Compound 4 (Yield 42%).

¹H-NMR (500 MHz, DMSO-d₆): δ 9.84 (s, 1H), 9.55 (s, 1H), 8.99 (s, 1H), 8.46 (s, 2H), 8.46-8.37 (m, 1H), 7.98 (s, 2H), 7.77 (s, 1H), 7.68-7.64 (m, 1H), 7.62 (s, 2H), 7.55-7.52 (m, 1H)

### 5. Preparation of Compound 5

The synthesis scheme of Compound 5 is shown in Scheme 5 below:

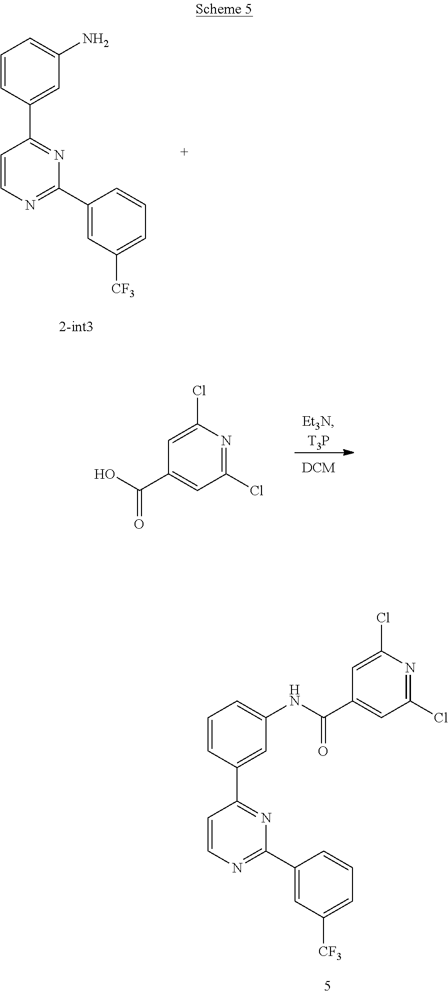 [SMILES: Nc1cccc(-c2ccnc(-c3cccc(C(F)(F)F)c3)n2)c1 ; O=C(O)c1cc(Cl)nc(Cl)c1 ; O=C(Nc1cccc(-c2ccnc(-c3cccc(C(F)(F)F)c3)n2)c1)c1cc(Cl)nc(Cl)c1]

16 mg of 2-int3 was dissolved in 1 mL of DCM to form a solution, and 1 eq of 2,6-dichloropyridine-4-carboxylic acid, 3 eq of Et₃N and 1.5 eq of T3P were added to the solution. Next, the solution was stirred at room temperature overnight. After the reaction was completed, the resulting solution was added to water and extracted with DCM. After the extraction, the DCM layer was taken out. The DCM layer was dried, concentrated, and purified with a column to obtain 12 mg of Compound 5 (Yield 42%).

¹H-NMR (500 MHz, DMSO-d₆): δ 10.87 (s, 1H), 9.05 (s, 1H), 8.83-8.74 (m, 3H), 8.09-8.00 (m, 5H), 7.95 (d, J=7 Hz, 1H), 7.85 (d, J=8 Hz, 1H), 7.63 (t, J=8 Hz, 1H)

### 6. Preparation of Compound 6

The synthesis scheme of Compound 6 is shown in Scheme 6 below:

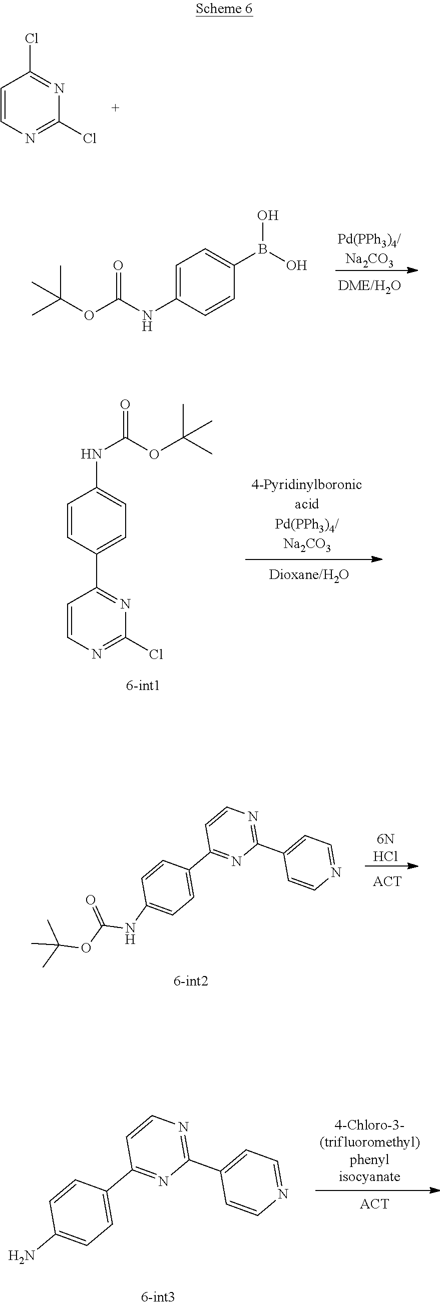

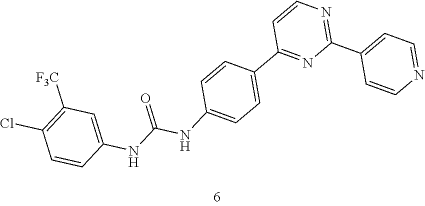 [SMILES: O=C(Nc1ccc(-c2ccnc(-c3ccncc3)n2)cc1)Nc1ccc(Cl)c(C(F)(F)F)c1]

(1) Preparation of Intermediate 6-int1

613 mg (1.0 eq) of 2,4-dichloropyrimidine was placed in a reaction flask under N₂, and 1.0 eq of 4-(N-Boc-amino)phenylboronic acid, 2.5 eq of Na₂CO₃ saturated aqueous solution and 20 mL of 1,2-dimethoxyethane were added to the reaction flask, separately. Next, after 4.5% mole Pd(PPh₃)₄ was added to the reaction flask, the solution in the reaction flask was refluxed and stirred for 2 hours. After the completion of the reaction was confirmed by thin layer chromatography (TLC) (hexane/EA=8:1), 1,2-dimethoxyethane was removed from the solution in the reaction flask under reduced pressure. After that, the solution in reaction flask was added to water and extracted with EA. After the extraction, the EA layer was taken out. The EA layer was dried, concentrated, and purified with a column to obtain 510 mg of 6-int1 (42%).

(2) Preparation of Intermediate 6-int2

100 mg (1.0 eq) of 6-int1 was placed in a reaction flask under N₂, and 1.0 eq of 4-pyridinylboronic acid, 2.5 eq of Na₂CO₃ saturated aqueous solution and 5 mL 1,4-dioxane were added to the reaction flask, separately. Next, after 4.5% mole Pd(PPh₃)₄ was added to the reaction flask, the solution in the reaction flask was refluxed and stirred for 2 hours. After the completion of the reaction was confirmed by thin layer chromatography (TLC) (hexane/EA=5:1), 1,4-dioxane was removed from the solution in the reaction flask under reduced pressure. After that, the solution in reaction flask was added to water and extracted with EA. After the extraction, the EA layer was taken out. The EA layer was dried, concentrated, and purified with a column to obtain 66 mg of 6-int2 (47%).

(3) Preparation of Intermediate 6-int3

66 mg of 6-int2 was completely dissolved in 2 mL of ACT to form a solution, and 6N HCl was added to the solution to react for 3 hours. After the reaction was completed, the liquid of the reacted reactant was sucked to dry, and then saturated NaHCO₃ and DCM were added for performing extraction. After the extraction, the organic layer was taken out. The organic layer was dried and concentrated to obtain 31 mg of 6-int3 (66%).

### (4) Preparation of Compound 6

16 mg of 6-int3 was dissolved in 1 mL of ACT to form a solution, and 1 eq of 4-chloro-3-(trifluoromethyl)phenyl isocyanate was added to the solution. Next, the solution was stirred at room temperature overnight. After the reaction was completed, the resulting solution was filtered to obtain a solid. The obtained solid was oven-dried to obtain 20 mg of Compound 6 (Yield 60%).

¹H-NMR (500 MHz, DMSO-d₆): δ 10.01 (s, 1H), 8.96 (d, J=6 Hz, 1H), 8.79 (d, J=6 Hz, 2H), 8.38 (d, J=5.5 Hz, 2H), 8.33 (d, J=9 Hz, 2H), 8.15 (s, 1H), 8.06(d, J=5 Hz, 1H), 7.73-7.7(m, 2H), 7.61 (d, J=9 Hz, 1H)

### 7. Preparation of Compound 7

The synthesis scheme of Compound 7 is shown in Scheme 7 below:

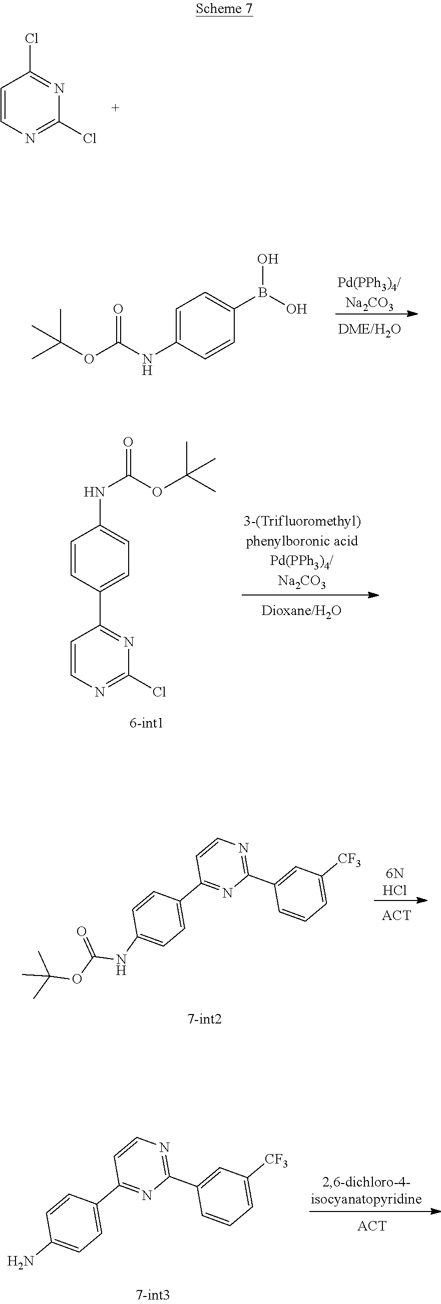

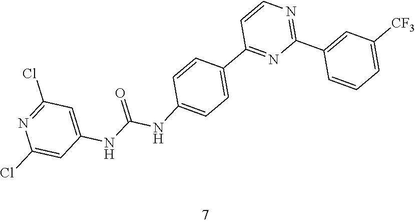 [SMILES: O=C(Nc1ccc(-c2ccnc(-c3cccc(C(F)(F)F)c3)n2)cc1)Nc1cc(Cl)nc(Cl)c1]

(1) Preparation of Intermediate 6-int1

The preparation method is the same as the preparation method for Intermediate 6-int1 in the preparation of Compound 6.

(2) Preparation of Intermediate 7-int2

100 mg (1.0 eq) of 6-int1 was placed in a reaction flask under N₂, and 1.0 eq of 3-(trifluoromethyl)phenylboronic acid, 2.5 eq of Na₂CO₃ saturated aqueous solution and 5 mL 1,4-dioxane were added to the reaction flask, separately. Next, after 4.5% mole Pd(PPh₃)₄ was added to the reaction flask, the solution in the reaction flask was refluxed and stirred for 2 hours. After the completion of the reaction was confirmed by thin layer chromatography (TLC) (hexane/EA=5:1), 1,4-dioxane was removed from the solution in the reaction flask under reduced pressure. After that, the solution in reaction flask was added to water and extracted with EA. After the extraction, the EA layer was taken out. The EA layer was dried, concentrated, and purified with a column to obtain 75 mg of 7-int2 (43%).

(3) Preparation of Intermediate 7-int3

75 mg of 7-int2 was completely dissolved in 2 mL of ACT to form a solution, and 6N HCl was added to the solution to react for 3 hours. After the reaction was completed, the liquid of the reacted reactant was sucked to dry, and then saturated NaHCO₃ and DCM were added for performing extraction. After the extraction, the organic layer was taken out. The organic layer was dried and concentrated to obtain 50 mg of 7-int3 (88%).

### (4) Preparation of Compound 7

20 mg of 7-int3 was dissolved in 1 mL of ACT to form a solution, and 1 eq of 2,6-dichloro-4-isocyanatopyridine was added to the solution. Next, the solution was stirred at room temperature overnight. After the reaction was completed, the resulting solution was filtered to obtain a solid. The obtained solid was oven-dried to obtain 18 mg of Compound 7 (Yield 59%).

¹H-NMR (500 MHz, DMSO-d₆): δ 9.72 (s, 1H), 9.58 (s, 1H), 8.95 (d, J=5.5 Hz, 1H), 8.82 (d, J=8 Hz, 1H), 8.76 (s, 1H), 8.34 (d, J=9 Hz, 2H), 8.03 (d, J=5 Hz, 1H), 7.93 (d, J=8 Hz, 1H), 7.84-7.80 (m, 1H), 7.70(d, J=9 Hz, 2H), 7.58 (s, 2H)

### 8. Preparation of Compound 8

The synthesis scheme of Compound 8 is shown in Scheme 8 below:

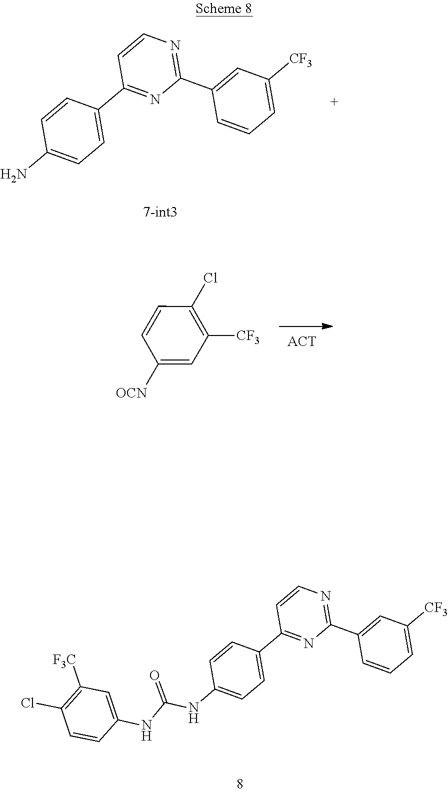

16 mg of 7-int3 was dissolved in 1 mL of ACT to form a solution, and 1 eq of 4-chloro-3-(trifluoromethyl)phenyl isocyanate was added to the solution. Next, the solution was stirred at room temperature overnight. After the reaction was completed, the resulting solution was filtered to obtain a solid. The obtained solid was oven-dried to obtain 10 mg of Compound 8 (Yield 30%).

¹H-NMR (500 MHz, DMSO-d₆): δ 9.02 (d, J=5 Hz, 1H), 9.84-9.81 (m, 2H), 8.59 (s, 1H), 8.17 (s, 1H), 8.05-7.96 (m, 1H), 7.94-7.91 (m, 1H), 8.05-7.96 (m, 1H), 7.80-7.73 (m, 2H), 7.70 (S, 1H), 7.61-7.59 (m, 1H), 7.52-7.50 (m, 1H)

### 9. Preparation of Compound 9

The synthesis scheme of Compound 9 is shown in Scheme 9 below:

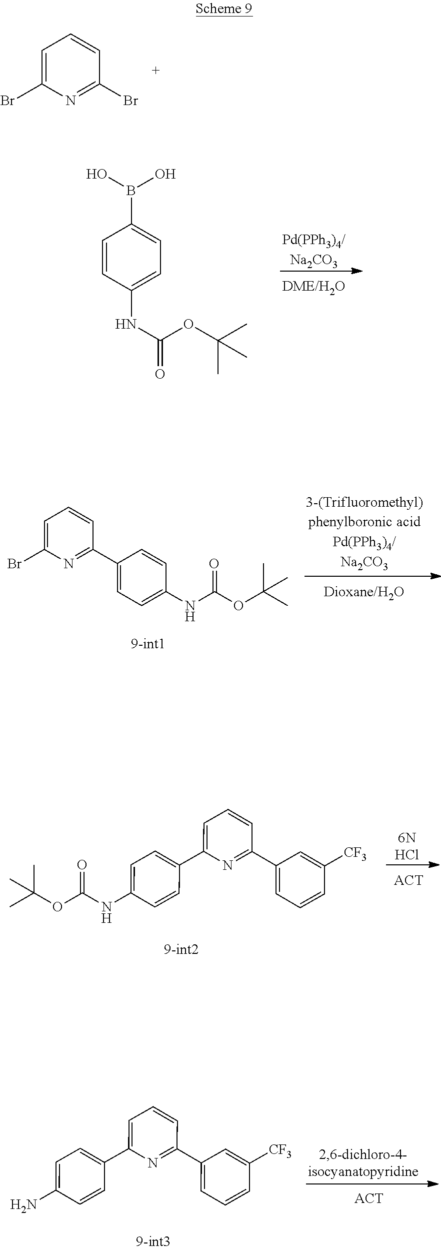

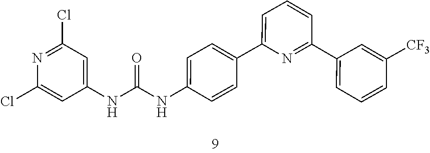 [SMILES: O=C(Nc1ccc(-c2cccc(-c3cccc(C(F)(F)F)c3)n2)cc1)Nc1cc(Cl)nc(Cl)c1]

(1) Preparation of Intermediate 9-int1

2.3 g (1.0 eq) of 2,6-dibromopyridine was placed in a reaction flask under N₂, and 1.0 eq of 4-(N-Boc-amino)phenylboronic acid, 2.5 eq of Na₂CO₃ saturated aqueous solution and 20 mL of 1,2-dimethoxyethane were added to the reaction flask, separately. Next, after 4.5% mole Pd(PPh₃)₄ was added to the reaction flask, the solution in the reaction flask was refluxed and stirred for 2 hours. After the completion of the reaction was confirmed by thin layer chromatography (TLC) (hexane/EA=8:1), 1,2-dimethoxyethane was removed from the solution in the reaction flask under reduced pressure. After that, the solution in reaction flask was added to water and extracted with EA. After the extraction, the EA layer was taken out. The EA layer was dried, concentrated, and purified with a column to obtain 1 g of 9-int1 (30%).

(2) Preparation of Intermediate 9-int2

1 g (1.0 eq) of 9-int1 was placed in a reaction flask under N₂, and 1.0 eq of 3-(trifluoromethyl)phenylboronic acid, 2.5 eq of Na₂CO₃ saturated aqueous solution and 20 mL of 1,4-dioxane were added to the reaction flask, separately. Next, after 4.5% mole Pd(PPh₃)₄ was added to the reaction flask, the solution in the reaction flask was refluxed and stirred for 2 hours. After the completion of the reaction was confirmed by thin layer chromatography (TLC) (hexane/EA=1:1), 1,4-dioxane was removed from the solution in the reaction flask under reduced pressure. After that, the solution in reaction flask was added to water and extracted with EA. After the extraction, the EA layer was taken out. The EA layer was dried, concentrated, and purified with a column to obtain 0.8 mg of 9-int2 (67%).

(3) Preparation of Intermediate 9-int3

0.8 g of 9-int2 was completely dissolved in 1 mL of ACT to form a solution, and 10 mL of 6N HCl was added to the solution to react for 3 hours. After the reaction was completed, the liquid of the reacted reactant was sucked to dry, and then saturated NaHCO₃ and DCM were added for performing extraction. After the extraction, the organic layer was taken out. The organic layer was dried and concentrated to obtain 0.5 g of 9-int3 (90%).

### (4) Preparation of Compound 9

15 mg of 9-int3 was dissolved in 1 mL of ACT to form a solution, and 1 eq of 2,6-dichloro-4-isocyanatopyridine was added to the solution. Next, the solution was stirred at room temperature overnight. After the reaction was completed, the resulting solution was filtered to obtain a solid. The obtained solid was oven-dried to obtain 5 mg of Compound 9 (Yield 22%).

¹H-NMR (500 MHz, DMSO-d₆): δ 8.40(s, 1H), 8.30 (d, J=8 Hz, 1H), 8.13 (d, J=8 Hz, 2H), 7.87 (t, J=7.5 Hz, 1H), 7.73-7.71 (m, 3H), 7.69-7.63 (m, 1H), 7.50 (d, J=8 Hz, 2H), 7.50 (d, J=8 Hz, 2H), 7.39 (s, 2H), 7.31 (s, 1H), 7.02 (s, 1H)

### 10. Preparation of Compound 10

The synthesis scheme of Compound 10 is shown in Scheme 10 below:

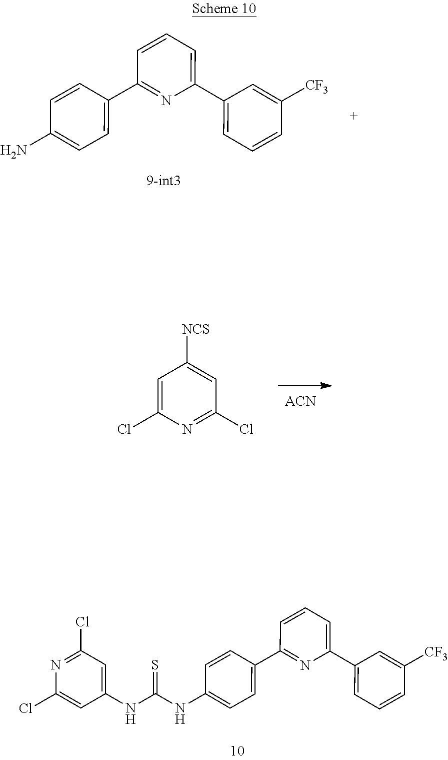

15 mg of 9-int3 was dissolved in 1 mL of ACT to form a solution, and 1 eq of 2,6-dichloro-4-isothiocyanatopyridine was added to the solution. Next, the solution was stirred at room temperature overnight. After the reaction was completed, the resulting solution was filtered to obtain a solid. The obtained solid was oven-dried to obtain 5 mg of Compound 10 (Yield 22%).

¹H-NMR (500 MHz, DMSO-d₆): δ 8.40(s, 1H), 8.33-8.29 (m, 3H), 8.04 (s, 1H), 7.93 (t, J=7.5 Hz, 1H), 7.79-7.78 (m, 2H), 7.77-7.63 (m, 5H), 7.44-7.43 (m, 2H)

### 11. Preparation of Compound 11

The synthesis scheme of Compound 11 is shown in Scheme 11 below:

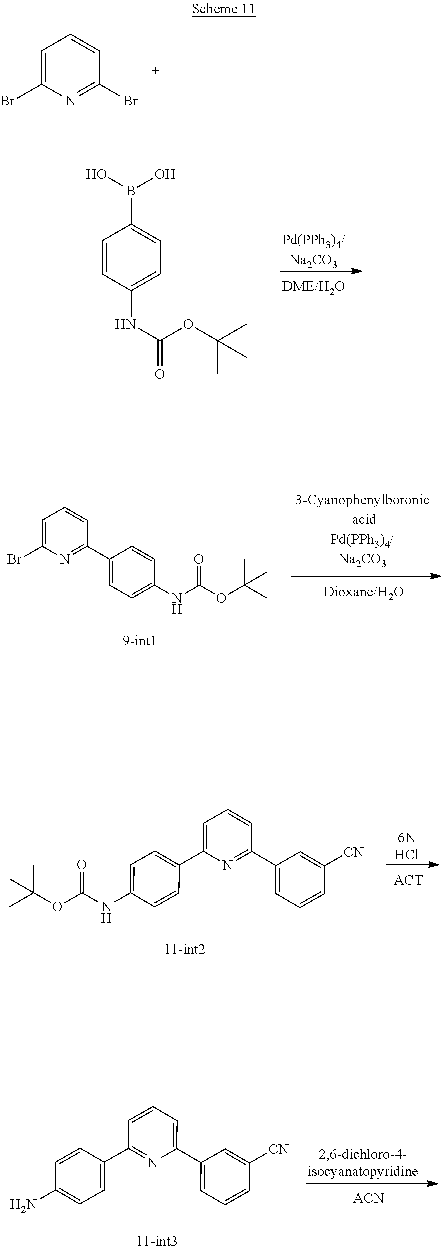

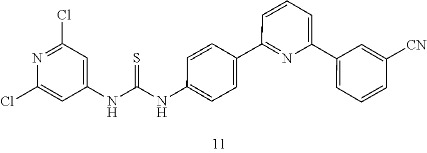 [SMILES: N#Cc1cccc(-c2cccc(-c3ccc(NC(=S)Nc4cc(Cl)nc(Cl)c4)cc3)n2)c1]

(1) Preparation of Intermediate 9-int1

The preparation method is the same as the preparation method for Intermediate 9-int1 in the preparation of Compound 9.

(2) Preparation of Intermediate 11-int2

80 mg (1.0 eq) of 9-int1 was placed in a reaction flask under N₂, and 1.0 eq of 3-cyanophenylboronic acid, 2.5 eq of Na₂CO₃ saturated aqueous solution and 2 mL of 1,4-dioxane were added to the reaction flask, separately. Next, after 4.5% mole Pd(PPh₃)₄ was added to the reaction flask, the solution in the reaction flask was refluxed and stirred for 2 hours. After the completion of the reaction was confirmed by thin layer chromatography (TLC) (hexane/EA=1:1), 1,4-dioxane was removed from the solution in the reaction flask under reduced pressure. After that, the solution in reaction flask was added to water and extracted with EA. After the extraction, the EA layer was taken out. The EA layer was dried, concentrated, and purified with a column to obtain 60 mg of 11-int2 (70%).

(3) Preparation of Intermediate 11-int3

60 mg of 11-int2 was completely dissolved in 20 mL of ACT to form a solution, and 2 mL of 6N HCl was added to the solution to react for 3 hours. After the reaction was completed, the liquid of the reacted reactant was sucked to dry, and then saturated NaHCO₃ and DCM were added for performing extraction. After the extraction, the organic layer was taken out. The organic layer was dried and concentrated to obtain 30 mg of 11-int3 (69%).

### (4) Preparation of Compound 11

13 mg of 11-int3 was dissolved in 1 mL of ACT to form a solution, and 1 eq of 2,6-dichloro-4-isothiocyanatopyridine was added to the solution. Next, the solution was stirred at room temperature overnight. After the reaction was completed, the resulting solution was filtered to obtain a solid. The obtained solid was oven-dried to obtain 5 mg of Compound 11 (Yield 22%).

¹H-NMR (500 MHz, DMSO-d₆): δ 8.65 (s, 1H), 8.57 (d, J=8.5 Hz, 1H), 8.23 (s, 2H), 8.03-7.92 (m, 4H), 7.76-7.67 (m, 2H)

### 12. Preparation of Compound 12

The synthesis scheme of Compound 12 is shown in Scheme 8 below:

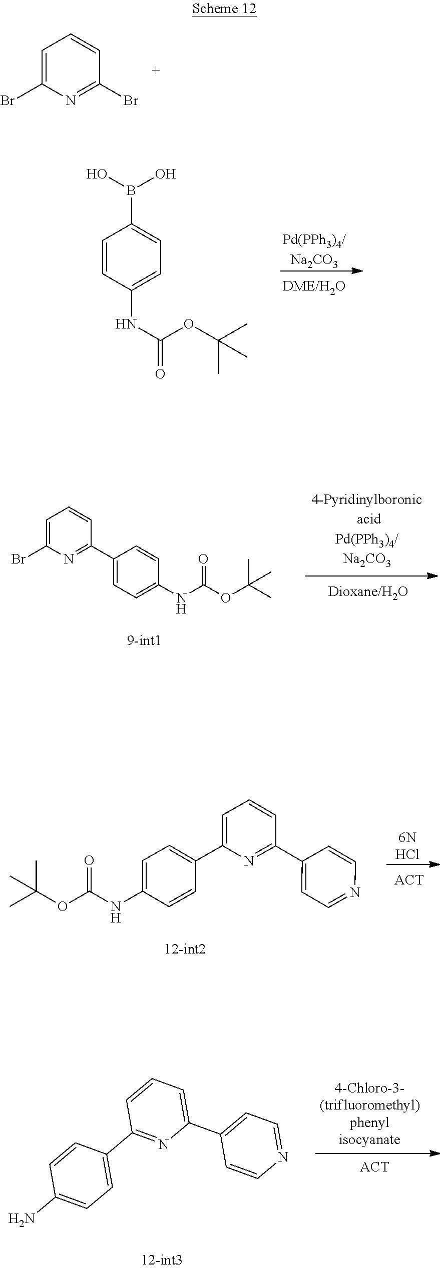

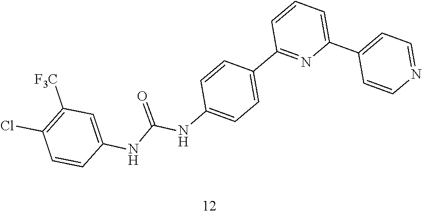 [SMILES: O=C(Nc1ccc(-c2cccc(-c3ccncc3)n2)cc1)Nc1ccc(Cl)c(C(F)(F)F)c1]

(1) Preparation of Intermediate 9-int1

The preparation method is the same as the preparation method for Intermediate 9-int1 in the preparation of Compound 9.

(2) Preparation of Intermediate 12-int2

42 mg (1.0 eq) of 9-int1 was placed in a reaction flask under N₂, and 1.0 eq of 4-pyridinylboronic acid, 2.5 eq of Na₂CO₃ saturated aqueous solution and 2 mL of 1,4-dioxane were added to the reaction flask, separately. Next, after 4.5% mole Pd(PPh₃)₄ was added to the reaction flask, the solution in the reaction flask was refluxed and stirred for 2 hours. After the completion of the reaction was confirmed by thin layer chromatography (TLC) (hexane/EA=1:1), 1,4-dioxane was removed from the solution in the reaction flask under reduced pressure. After that, the solution in reaction flask was added to water and extracted with EA. After the extraction, the EA layer was taken out. The EA layer was dried, concentrated, and purified with a column to obtain 20 mg of 12-int2 (41%).

(3) Preparation of Intermediate 12-int3

20 mg of 12-int2 was completely dissolved in 20 mL of ACT to form a solution, and 2 mL of 6N HCl was added to the solution to react for 3 hours. After the reaction was completed, the liquid of the reacted reactant was sucked to dry, and then saturated NaHCO₃ and DCM were added for performing extraction. After the extraction, the organic layer was taken out. The organic layer was dried and concentrated to obtain 11 mg of 12-int3 (77%).

### (4) Preparation of Compound 12

11 mg of 12-int3 was dissolved in 1 mL of ACT to form a solution, and 1 eq of 4-chloro-3-(trifluoromethyl)phenyl isocyanate was added to the solution. Next, the solution was stirred at room temperature overnight. After the reaction was completed, the resulting solution was filtered to obtain a solid. The obtained solid was oven-dried to obtain 10 mg of Compound 12 (Yield 48%).

¹H-NMR (500 MHz, DMSO-d₆): δ 9.40 (S, 1H), 9.41 (S, 1H), 8.73 (d, J=6 Hz, 2H), 8.19-8.12 (m, 5H), 8.02-8.01 (m, 3H), 7.68-7.60 (m, 4H)

### 13. Preparation of Compound 13

The synthesis scheme of Compound 13 is shown in Scheme 13 below:

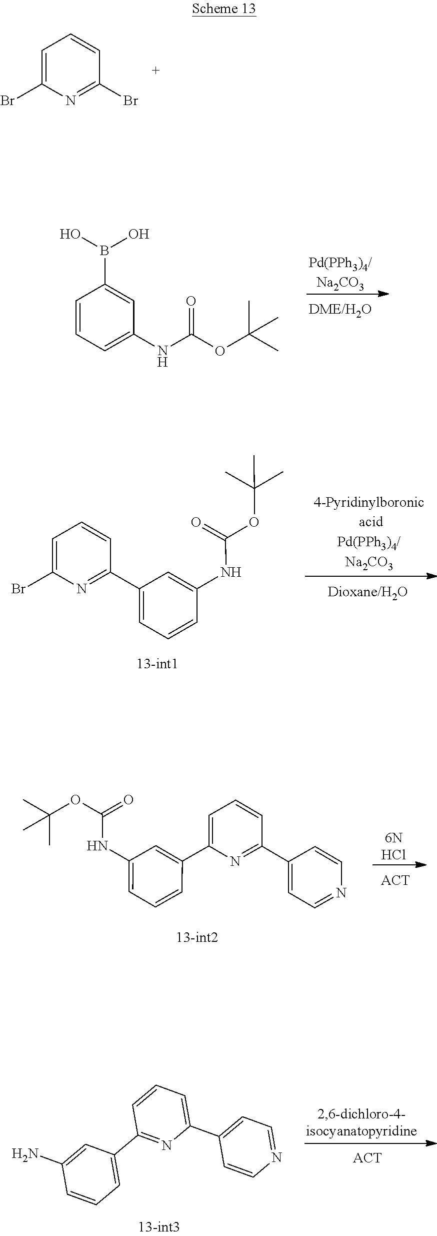

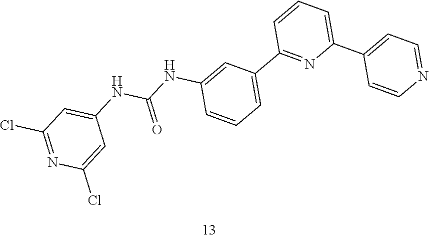 [SMILES: O=C(Nc1cccc(-c2cccc(-c3ccncc3)n2)c1)Nc1cc(Cl)nc(Cl)c1]

(1) Preparation of Intermediate 13-int1

2.3 g (1.0 eq) of 2,6-dibromopyridine was placed in a reaction flask under N₂, and 1.0 eq of 3-(N-Boc-amino)phenylboronic acid, 2.5 eq of Na₂CO₃ saturated aqueous solution and 20 mL of 1,2-dimethoxyethane were added to the reaction flask, separately. Next, after 4.5% mole Pd(PPh₃)₄ was added to the reaction flask, the solution in the reaction flask was refluxed and stirred for 2 hours. After the completion of the reaction was confirmed by thin layer chromatography (TLC) (hexane/EA=5:1), 1,2-dimethoxyethane was removed from the solution in the reaction flask under reduced pressure. After that, the solution in reaction flask was added to water and extracted with EA. After the extraction, the EA layer was taken out. The EA layer was dried, concentrated, and purified with a column to obtain 1 g of 13-int1 (30%).

(2) Preparation of Intermediate 13-int2

420 mg (1.0 eq) of 13-int1 was placed in a reaction flask under N₂, and 1.0 eq of 4-pyridinylboronic acid, 2.5 eq of Na₂CO₃ saturated aqueous solution and 2 mL of 1,4-dioxane were added to the reaction flask, separately. Next, after 4.5% mole Pd(PPh₃)₄ was added to the reaction flask, the solution in the reaction flask was refluxed and stirred for 2 hours. After the completion of the reaction was confirmed by thin layer chromatography (TLC) (hexane/EA=1:1), 1,4-dioxane was removed from the solution in the reaction flask under reduced pressure. After that, the solution in reaction flask was added to water and extracted with EA. After the extraction, the EA layer was taken out. The EA layer was dried, concentrated, and purified with a column to obtain 300 mg of 13-int2 (60%).

(3) Preparation of Intermediate 13-int3

100 mg of 13-int2 was completely dissolved in 2 mL of ACT to form a solution, and 2 mL of 6N HCl was added to the solution to react for 3 hours. After the reaction was completed, the liquid of the reacted reactant was sucked to dry, and then saturated NaHCO₃ and DCM were added for performing extraction. After the extraction, the organic layer was taken out. The organic layer was dried and concentrated to obtain 70 mg of 13-int3 (99%).

### (4) Preparation of Compound 13

11 mg of 13-int3 was dissolved in 1 mL of ACT to form a solution, and 1 eq of 2,6-dichloro-4-isothiocyanatopyridine was added to the solution. Next, the solution was stirred at room temperature overnight. After the reaction was completed, the resulting solution was filtered to obtain a solid. The obtained solid was oven-dried to obtain 9 mg of Compound 13 (Yield 46%).

¹H-NMR (500 MHz, DMSO-d₆): δ 9.74(S, 1H), 9.42 (S, 1H), 8.74 (d, J=6 Hz, 2H), 8.29(S,1H), 8.18-8.17 (m, 2H), 8.11-8.08 (m, 2H), 8.01-7.99 (m, 1H), 7.86 (d, J=7.5 Hz, 1H), 7.66 (d, J=8 Hz, 1H), 7.50(S,2H), 7.50-7.46 (m, 1H)

### 14. Preparation of Compound 14

The synthesis scheme of compound 14 is shown in Scheme 14 below:

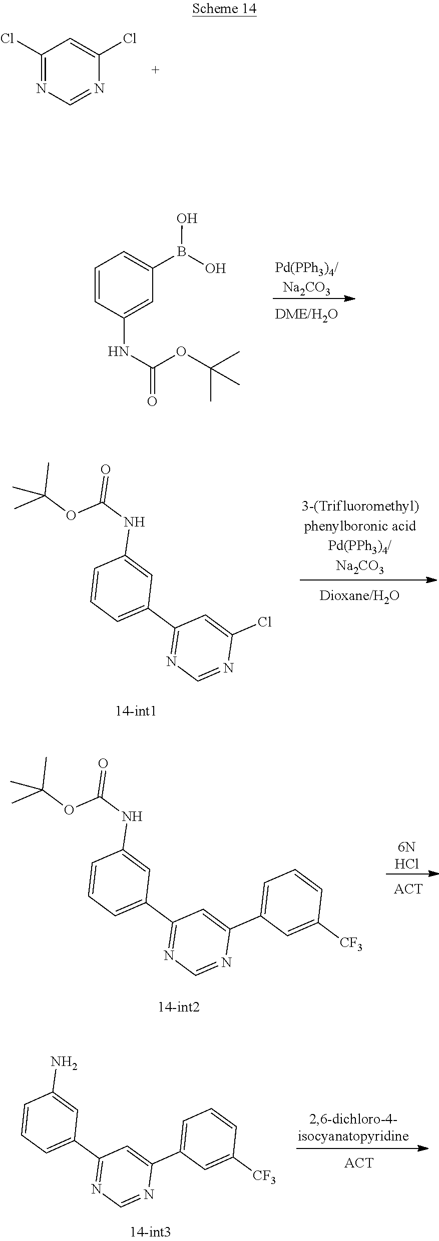

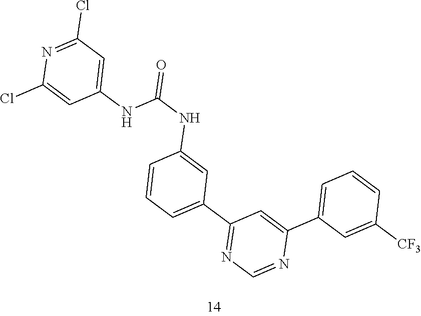 [SMILES: O=C(Nc1cccc(-c2cc(-c3cccc(C(F)(F)F)c3)ncn2)c1)Nc1cc(Cl)nc(Cl)c1]

(1) Preparation of Intermediate 14-int1

500 mg (1.0 eq) of 4,6-dichloropyrimidine was placed in a reaction flask under N₂, and 1.0 eq of 3-(N-Boc-amino)phenylboronic acid, 2.5 eq of Na₂CO₃ saturated aqueous solution and 20 mL of 1,2-dimethoxyethane were added to the reaction flask, separately. Next, after 4.5% mole Pd(PPh₃)₄ was added to the reaction flask, the solution in the reaction flask was refluxed and stirred for 2 hours. After the completion of the reaction was confirmed by thin layer chromatography (TLC) (hexane/EA=5:1), 1,2-dimethoxyethane was removed from the solution in the reaction flask under reduced pressure. After that, the solution in reaction flask was added to water and extracted with EA. After the extraction, the EA layer was taken out. The EA layer was dried, concentrated, and purified with a column to obtain 140 mg of 14-int1 (30%).

(2) Preparation of Intermediate 14-int2

70 mg (1.0 eq) of 14-int1 was placed in a reaction flask under N₂, and 1.0 eq of 3-(trifluoromethyl)phenylboronic acid, 2.5 eq of Na₂CO₃ saturated aqueous solution and 20 mL of 1,4-dioxane were added to the reaction flask, separately. Next, after 4.5% mole Pd(PPh₃)₄ was added to the reaction flask, the solution in the reaction flask was refluxed and stirred for 2 hours. After the completion of the reaction was confirmed by thin layer chromatography (TLC) (hexane/EA=1:1), 1,4-dioxane was removed from the solution in the reaction flask under reduced pressure. After that, the solution in reaction flask was added to water and extracted with DCM. After the extraction, the DCM layer was taken out. The DCM layer was dried, concentrated, and purified with a column to obtain 36 mg of 14-int2 (43%).

(3) Preparation of Intermediate 14-int3

36 mg of 14-int2 was completely dissolved in 1 mL of ACT to form a solution, and 1 mL of 6N HCl was added to the solution to react for 3 hours. After the reaction was completed, the liquid of the reacted reactant was sucked to dry, and then saturated NaHCO₃ and DCM were added for performing extraction. After the extraction, the organic layer was taken out. The organic layer was dried and concentrated to obtain 17 mg of 14-int3 (68%).

### (4) Preparation of Compound 14

15 mg of 9-int3 was dissolved in 1 mL of ACT to form a solution, and 1 eq of 2,6-dichloro-4-isocyanatopyridine was added to the solution. Next, the solution was stirred at room temperature overnight. After the reaction was completed, the resulting solution was filtered to obtain a solid. The obtained solid was oven-dried to obtain 10 mg of Compound 14 (Yield 29%).

¹H-NMR (500 MHz, DMSO-d₆): δ 9.35(S, 1H), 8.70-8.66 (m, 3H), 8.51(S,1H), 8.07(S,1H), 7.94(S,1H), 7.83(S,1H), 7.68 (d, J=8.5 Hz, 1H), 7.67 (s, 2H), 7.60-7.52 (m, 1H)

### 15. Preparation of Compound 15

The synthesis scheme of Compound 15 is shown in Scheme 15 below:

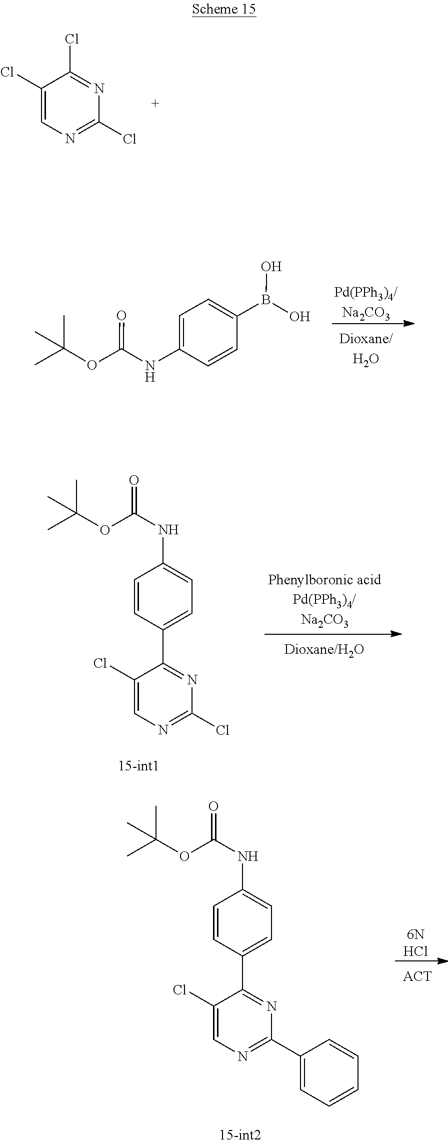 [SMILES: Clc1ncc(Cl)c(Cl)n1 ; CC(C)(C)OC(=O)Nc1ccc(B(O)O)cc1 ; CC(C)(C)OC(=O)Nc1ccc(-c2nc(Cl)ncc2Cl)cc1 ; CC(C)(C)OC(=O)Nc1ccc(-c2nc(-c3ccccc3)ncc2Cl)cc1 ; "6N HCl"]

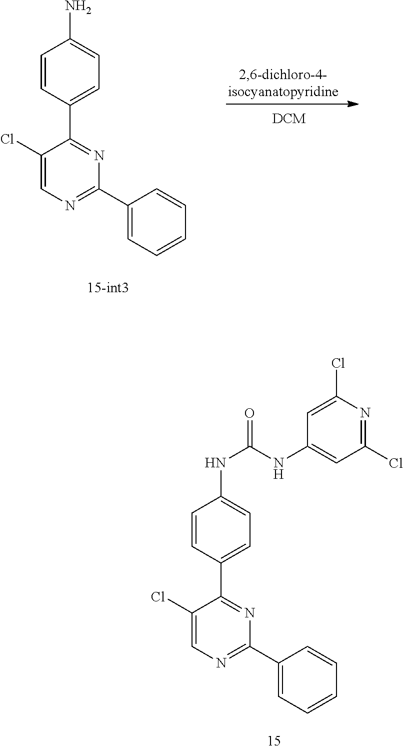 [SMILES: Nc1ccc(-c2nc(-c3ccccc3)ncc2Cl)cc1 ; O=C(Nc1ccc(-c2nc(-c3ccccc3)ncc2Cl)cc1)Nc1cc(Cl)nc(Cl)c1]

(1) Preparation of Intermediate 15-int1

800 mg (1.0 eq) of 2,4,5-trichloropyrimidine was placed in a reaction flask under N₂, and 1.0 eq of 4-(N-Boc-amino)phenylboronic acid, 2.5 eq of Na₂CO₃ saturated aqueous solution and 680 mL of 1,4-dioxane were added to the reaction flask, separately. Next, after 4.5% mole Pd(PPh₃)₄ was added to the reaction flask, the solution in the reaction flask was refluxed and stirred for 2 hours. After the completion of the reaction was confirmed by thin layer chromatography (TLC) (hexane/EA=5:1), 1,2-dimethoxyethane was removed from the solution in the reaction flask under reduced pressure. After that, the solution in reaction flask was added to water and extracted with DCM. After the extraction, the DCM layer was taken out. The DCM layer was dried, concentrated, and purified with a column to obtain 750 mg of 15-int1 (50%).

(2) Preparation of Intermediate 15-int2

750 mg (1.0 eq) of 15-int1 was placed in a reaction flask under N₂, and 1.0 eq of phenylboronic acid, 2.5 eq of Na₂CO₃ saturated aqueous solution and 400 mL of 1,4-dioxane were added to the reaction flask, separately. Next, after 4.5% mole Pd(PPh₃)₄ was added to the reaction flask, the solution in the reaction flask was refluxed and stirred for 5 hours. After the completion of the reaction was confirmed by thin layer chromatography (TLC) (hexane/EA=1:1), 1,4-dioxane was removed from the solution in the reaction flask under reduced pressure. After that, the solution in reaction flask was added to water and extracted with DCM. After the extraction, the DCM layer was taken out. The DCM layer was dried, concentrated, and then purified through recrystallization by MeOH to obtain 500 mg of 15-int2 (60%).

(3) Preparation of Intermediate 15-int3

500 mg of 15-int2 was completely dissolved in 1 mL of ACT to form a solution, and 100 mL of 6N HCl was added to the solution to react for 3 hours. ACT was added to the resulting solution to precipitate a solid, and then the resulting solution was filtered to obtain the precipitated solid. Saturated NaHCO₃ was added to the precipitated solid and stirred to form another solution. The other solution was filtered to obtain 300 mg of filter residue as 15-int3 (82%).

### (4) Preparation of Compound 15

260 mg of 15-int3 was dissolved in 200 mL of DCM to form a solution, and 25 g (1.2 eq) of 2,6-dichloro-4-isocyanatopyridine was added to the solution. Next, the solution stood at room temperature for 5 hours. After the reaction was completed, the resulting solution was filtered to obtain a solid. The obtained solid was purified through recrystallization by MeOH and oven-dried to obtain 300 mg of Compound 15 (Yield 71%).

¹H-NMR (500 MHz, DMSO-d₆): δ 9.69(S, 1H), 9.54(S, 1H), 9.02(S, 1H), 8.43-8.41 (m, 2H), 8.00 (d, J=8 Hz, 2H), 7.70-7.68 (m, 2H), 7.58-7.54 (m, 5H)

### C. Activity Analysis for Compounds

### C-1. Effect of Compounds on GNAQ Activity Displayed by Serum Response Element (SRE) Luciferase Reporter Gene-HEK293 Cell Line

### 1. Experimental Methods

7.5×10⁴ SRE-293-GNAQ (WT) or SRE-293-GNAQ (T96S) cell lines were seeded in a 24-well plate and cultured in a carbon dioxide incubator at 37° C. SRE-293-GNAQ (WT) or SRE-293-GNAQ (T96S) cell lines was derived from additionally transfecting SRE Reporter—HEK293 Recombinant Cell Line (ERK Pathway) (Catalog number: 60406; BPS Bioscience) cells with an expression vector separately expressing GNAQ (WT) or GNAQ (T96S), and screening a stable expression cell line by the antibiotic, geneticin (type number 10131035; Thermo Fisher Scientific). GNAQ (WT) or GNAQ (T96) expression vector was derived from gene synthesis and point mutation modification of GenScript company's customized services.

After 48 hours of culture, the culture medium in the 24-well plate was replaced with minimal essential medium (MEM) containing only 0.1% fetal bovine serum (FBS) and the cells were cultured overnight.

After overnight culture, the compound to be tested was added to the cells and reacted for 2 hours, and the medium at this point was still MEM medium containing only 0.1% fetal bovine serum. After reacting with the compound to be tested for 2 hours, fetal bovine serum at a final concentration of 20% was added to the cells and reacted for at least 4 hours. After the reaction, the supernatant was removed. Next, 100 μL of cell lysis reagent CelLytic M Cell Lysis Reagent (Catalog number: C2978; Sigma) was added to the cells and aspirated repeatedly several times to form a cell lysate solution with the cells. The cell lysate solution was collected in a microcentrifuge tube and centrifuged at a high speed of 14,000 rpm for 5 minutes. After that, 30 μL of supernatant was taken out and placed in a 96-well white plate for luminescence analysis, and 30 μL of firefly luminescence analysis reagent (Catalog No. E1501; Promega) diluted 3-fold was added and mixed well, and then luminescence detection was performed with a plate enzyme immunoassay analyzer (ELISA reader) to evaluate the effect of the compound to be tested on MAPK signaling induced by GNAQ through the expression of the firefly luciferase reporter gene.

### 2. Experimental Results

In order to confirm the effect of the compounds on MAPK signaling induced by GNAQ, the screened SRE-HEK293 cells capable of expressing exogenous GNAQ (WT) and GNAQ (T96S) genes were subjected to overnight serum starvation, and 2 hours before induction with 20% fetal bovine serum, the compound to be tested (one of Compound 1 to Compound 15 prepared above) was added to the cells, and after serum induction, the expression of the firefly luciferase reporter gene was measured. Through the above steps, the inhibition of the compound to be tested on GNAQ expression could be evaluated through the expression of the reporter gene in cells.

The results are shown in Table 1 below. Inhibition of the compounds on GNAQ expression is shown on a 3-level scale. +++ represents that the inhibitory rate is greater than 70%; ++ represents that the inhibitory rate is 70%˜40%; + represents that the inhibitory rate is 39%˜10% inhibitory rate. In addition, − represents that the inhibitory rate is less than 10%.

TABLE 1
Inhibition of compounds on GNAQ expression

| | GNAQ inhibitory rate | | | | | |
|---|---|---|---|---|---|---|
| | Cell type | | | | | |
| | WT | T96S | WT | T96S | WT | T96S |
| | Analytical concentration | | | | | |
| Compound | 8,100 nM | | 2,700 nM | | 900 nM | |
| 1 | +++ | +++ | + | + | − | − |
| 2 | +++ | +++ | + | + | + | − |
| 3 | +++ | +++ | + | ++ | + | − |
| 4 | ++ | ++ | + | + | − | − |
| 5 | − | − | + | − | − | − |
| 6 | + | ++ | + | + | − | + |
| 7 | + | + | − | + | − | − |
| 8 | − | + | − | − | − | − |
| 9 | +++ | ++ | + | + | + | − |
| 10 | +++ | +++ | ++ | ++ | + | − |
| 11 | +++ | +++ | ++ | ++ | + | + |
| 12 | ++ | ++ | + | ++ | + | + |
| 13 | +++ | +++ | ++ | ++ | + | + |
| 14 | ++ | +++ | + | + | − | + |
| 15 | +++ | ++ | + | + | − | − |

According to Table 1 above, it is known that Compound 1 through Compound 15 all have inhibitory effects on GNAQ expression.

C-2. Analysis of Inhibition of Compounds on Growth of Mammary Gland Cancer Cells (Half Maximal Inhibitory Concentration (IC₅₀) Analysis) and Analysis of Half Cytotoxic Concentration (50% Cytotoxic Concentration, CC₅₀) of Compounds on Normal Mammary Gland Epithelial Cells

### 1. Experimental Methods

### (1) Analysis of Inhibition of Compounds on Growth of Human Mammary Gland Cancer Cells (Half Maximal Inhibitory Concentration (IC₅₀) Analysis)

Human mammary gland cancer cells, MCF-7 cells and MCF-7 GNAQ (T96S) cells (MCF-7 cells expressing GNAQ (T96S) after GNAQ gene deletion) were cultured in MEM medium.

1×10³ of the cells mentioned above were seeded in a 96-well plate and cultured overnight (at least 16 hours) in a carbon dioxide incubator.

After overnight incubation, the supernatant was removed. Next, after 200 μL of cell culture medium (containing 2% fetal bovine serum) containing different concentrations of test compound was added to each well of the 96-well plate, the 96-well plate was placed in a 5% carbon dioxide incubator at 37° C. for 7 days further culture. After that, an additional 30 μL of diluted PrestoBlue™ Cell Viability Reagent (Catalog Number: A13261; Thermo Fisher Scientific) (4 μL stock solution and 26 μL culture medium) was added to each well of the 96-well plate and the reaction was continued for further about 1 hour.

Finally, the fluorescence value at the wavelength of 535/615 nm of each well was read with a plate enzyme immunoassay analyzer, and the inhibitory rate of the compound on mammary gland cancer cells were calculated using the following formula.

$$Cell inhibitory rate ( \% ) = [ 1 - ( fluorescence value of experimental group - fluorescence value of blank group ) / ( fluorescence value of control group - fluorescence value of blank group ) ] \times 100$$

Moreover, the half maximal inhibitory concentration (IC₅₀) of the compound on the growth of mammary gland cancer cells was calculated with the inhibitory rate of the compound on mammary gland cancer cells at each concentration through the online half maximal inhibitory concentration (IC₅₀) calculation software, IC₅₀ Calculator, provided by AAT Bioquest company on its website (https://www.aatbio.com/tools/ic50-calculator).

Finally, the inhibition of the compound on the growth of mammary gland cancer cells was evaluated by the calculated half maximal inhibitory concentration (IC₅₀) of the compound on the growth of mammary gland cancer cells.

### (2) Analysis of Half Cytotoxic Concentration (50% Cytotoxic Concentration, CCso) of Compounds on Human Normal Mammary Gland Epithelial Cells

Human normal mammary epithelial cells, MCF-10A-5E cells, were cultured in DMEM/F12K (1:1) medium, wherein Insulin-Transferrin-Selenium (ITS-G) (41400045; Thermo Fisher Scientific) diluted 1,000-fold for use, EGF stock (1 mg/mL) (10605-HNAE; Sino Biological) diluted 2,000-fold for use and Hydrocortisone stock (1 mg/mL) (S1696; Selleck Chemicals) diluted 2,000-fold for use were additionally added to the medium.

5×10³ of the cells mentioned above were seeded in a 96-well plate and cultured overnight (at least 16 hours) in a carbon dioxide incubator.

After overnight incubation, the supernatant was removed. Next, after 200 μL of cell culture medium (containing 10% horse serum) containing different concentrations of test compound was added to each well of the 96-well plate, the 96-well plate was placed in a 5% carbon dioxide incubator at 37° C. for 5 days further culture. After that, an additional 30 μL of diluted PrestoBlue™ Cell Viability Reagent (Catalog Number: A13261; Thermo Fisher Scientific) (4 μL stock solution and 26 μL culture medium) was added to each well of the 96-well plate and the reaction was continued for further about 1 hour.

Finally, the fluorescence value at the wavelength of 535/615 nm of each well was read with a plate enzyme immunoassay analyzer, and the fatality rate of the compound on normal mammary gland epithelial cells were calculated using the following formula.

$$Cell fatality rate ( \% ) = [ 1 - ( fluorescence value of experimental group - fluorescence value of blank group ) / ( fluorescence value of control group - fluorescence value of blank group ) ] \times 100$$

Moreover, the half cytotoxic concentration (50% cytotoxic concentration, CC₅₀) of the compound on normal mammary gland epithelial cells was calculated with the fatality rate of compound on normal mammary gland epithelial cells at each concentration through the online half maximal inhibitory concentration (IC₅₀) calculation software, ICso Calculator, provided by AAT Bioquest company on its website (https://www.aatbio.com/tools/ic50-calculator).

Finally, toxicity of the compound on normal mammary gland epithelial cells was evaluated by the calculated half cytotoxic concentration (CC₅₀) of the compound on normal mammary gland epithelial cells.

### 2. Experimental Results

The inhibition of the compound on the growth of breast cancer cells was evaluated by the half maximal inhibitory concentration (IC₅₀) of the compound (one of Compound 1 to Compound 15 prepared above) on the growth of mammary gland cancer cells through the human mammary gland cancer cell line MCF-7 and the human mammary gland cancer cell line MCF-7 GNAQ (T96S) expressing the mutated gene.

In addition, the toxicity of the compound on normal mammary gland epithelial cells was evaluated by the half cytotoxic concentration (CC₅₀) of the compound (one of Compound 1 to Compound 15 prepared above) on normal mammary gland epithelial cells through the human normal mammary gland epithelial cell line MCF-T0A-5E.

The results are shown in Table 2 below.

The intensity of the inhibitory activity of the compound on the growth of mammary gland cancer cells is shown with the symbol “+”. The more symbols “+” represent, the higher the inhibitory activity. +++ represents that the IC₅₀ of the compound on the growth of breast cancer cells is less than 0.31 μM; ++ represents that the IC₅₀ of the compound on the growth of breast cancer cells is 0.31˜2.5 μM; + represents that the IC₅₀ of the compound on the growth of breast cancer cells is 2.5˜5 μM. − represents that the IC₅₀ of the compound on the growth of breast cancer cells is greater than 5 μM.

The non-toxicity of the compound on the growth of normal mammary gland epithelial cells is shown with the symbol “+”, the more symbols “+” represent, the lower the toxicity. +++ represents that the CC₅₀ of the compound on the growth of breast cancer cells is greater than 20 μM; ++ represents that the CC₅₀ of the compound on the growth of breast cancer cells is 10˜20 μM; + represents that the CC₅₀ of the compound on the growth of breast cancer cells is 5˜10 μM. − represents that the CC₅₀ of the compound on the growth of breast cancer cells is less than 5 μM.

TABLE 2
Analysis for the inhibitory activity of compounds
on the growth of MCF GNAQ related cell lines and
the toxicity of compounds on MCF-10A-5E cells

| | | |
|---|---|---|
| | MCF-7 | |
| MCF-7 | GNAQ(T96S) | MCF-10A-5E |

| | | | |
|---|---|---|---|
| | | Inhibitory activity | Non-toxicity |
| Compound | Cell | (evaluated by IC₅₀) | (evaluated by CC₅₀) |

| | | | |
|---|---|---|---|
| 1 | ++ | ++ | +++ |
| 2 | ++ | ++ | ++ |
| 3 | ++ | ++ | ++ |
| 4 | ++ | ++ | + |
| 5 | ++ | ++ | +++ |
| 6 | ++ | ++ | ++ |
| 7 | ++ | ++ | ++ |
| 8 | ++ | ++ | ++ |
| 9 | ++ | ++ | ++ |
| 10 | ++ | ++ | +++ |
| 11 | ++ | ++ | +++ |
| 12 | ++ | ++ | ++ |
| 13 | ++ | ++ | +++ |
| 14 | +++ | +++ | ++ |
| 15 | +++ | +++ | ++ |

According to Table 2 above, it is known that Compound 1 through Compound 15 all have inhibitory effects on mammary gland cancer cells while are low toxic (or non-toxic) to the normal cells.

### C-3. Analysis of Inhibition of Compounds on Growth of Triple-Negative Breast Cancer Cells (Half Maximal Inhibitory Concentration (IC₅₀) Analysis):

### 1. Experimental Methods

Triple-negative breast cancer cells, MDA-MB-231 and MDA-MB-231-OE-GNAQ (T96S) (additionally expressing GNAQ (T96S)) were cultured in L-15 medium and in a temperature-controlled incubator without need of additional offer of 5% carbon dioxide.

1×10³ of the cells mentioned above were seeded in a 96-well plate, placed in a temperature-controlled incubator and cultured overnight (at least 16 hours).

After overnight incubation, the supernatant was removed. Next, after 200 μL of cell culture medium (containing 2% fetal bovine serum) containing different concentrations of test compound was added to each well of the 96-well plate, the 96-well plate was placed in a 37° C. incubator for 7 days further culture. After that, an additional 30 μL of diluted PrestoBlue™ Cell Viability Reagent (Catalog Number: A13261; Thermo Fisher Scientific) (4 μL stock solution and 26 μL culture medium) was added to each well of the 96-well plate and the reaction was continued for further about 1 hour.

Finally, the fluorescence value at the wavelength of 535/615 nm of each well was read with a plate enzyme immunoassay analyzer, and the inhibitory rate of the compound on triple-negative breast cancer cells were calculated using the following formula.

$$Cell inhibitory rate ( \% ) = [ 1 - ( fluorescence value of experimental group - fluorescence value of blank group ) / ( fluorescence value of control group - fluorescence value of blank group ) ] \times 100$$

Moreover, the half maximal inhibitory concentration (IC₅₀) of the compound on the growth of triple-negative breast cancer cells was calculated with the inhibitory rate of compound on triple-negative breast cancer cells at each concentration through the online half maximal inhibitory concentration (IC₅₀) calculation software, IC₅₀ Calculator, provided by AAT Bioquest company on its website (https://www.aatbio.com/tools/ic50-calculator).

Finally, the inhibition of the compound on the growth of triple-negative breast cancer cells was evaluated by the calculated half maximal inhibitory concentration (IC₅₀) of the compound on the growth of triple-negative breast cancer cells.

### 2. Experimental Results

The inhibition of the compound on the growth of triple-negative breast cancer cells was evaluated by the half maximal inhibitory concentration (IC₅₀) of the compound (one of Compound 1 to Compound 15 prepared above) on the growth of triple-negative breast cancer cells through the human triple-negative breast cancer cell lines MDA-MB-231 and MDA-MB-231-OE-GNAQ (T96S) (additionally expressing GNAQ (T96S)).

The results are shown in Table 3 below.

The intensity of the inhibitory activity of the compound on the growth of triple-negative breast cancer cells is shown with the symbol “+”. The more symbols “+” represent, the higher the inhibitory activity. +++ represents that the IC₅₀ of the compound on the growth of triple-negative breast cancer cells is less than 0.31 μM; ++ represents that the IC₅₀ of the compound on the growth of triple-negative breast cancer cells is 0.31-2.5 μM; + represents that the IC₅₀ of the compound on the growth of triple-negative breast cancer cells is 2.5-5 μM. − represents that the IC₅₀ of the compound on the growth of triple-negative breast cancer cells is greater than 5 μM.

TABLE 3
Analysis for the inhibitory activity of compounds on the growth
of MDA-MB-231 and MDA-MB-231-OE-GNAQ (T96S) cell lines

| | |
|---|---|
| MDA-MB-231 | MDA-MB-231-OE-GNAQ (T96S) |

| | | |
|---|---|---|
| Compound | Cell | Inhibitory activity (evaluated by IC₅₀) |

| | | |
|---|---|---|
| 1 | ++ | ++ |
| 2 | ++ | ++ |
| 3 | ++ | ++ |
| 4 | ++ | ++ |
| 5 | ++ | ++ |
| 6 | + | + |
| 7 | ++ | ++ |
| 8 | ++ | ++ |
| 9 | ++ | ++ |
| 10 | ++ | ++ |
| 11 | ++ | ++ |
| 12 | ++ | ++ |
| 13 | ++ | ++ |
| 14 | ++ | ++ |
| 15 | ++ | ++ |

According to Table 3 above, it is known that Compound 1 through Compound 15 all have excellent inhibitory effects on triple-negative breast cancer cells.

It will be apparent to those skilled in the art that various modifications and variations can be made to the disclosed embodiments. It is intended that the specification and examples be considered as exemplary only, with the true scope of the disclosure being indicated by the following claims and their equivalents.

What is claimed is:

1. A small molecule compound or salt thereof or solvates of them, wherein the small molecule compound comprises a compound represented by Formula (I) as shown below: 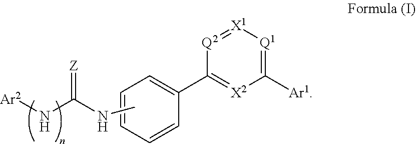
wherein, in Formula (I)
Q¹ is CR¹ or N,
Q² is CR² or N,
X¹ is CR³ or N, and
X² is CR⁴ or N,
wherein
R¹ and R² are independently H, alkyl, alkylhalo or halogen,
R³ and R⁴ are independently H or alkyl, and
when Q¹ and X² are both N, Q² is CR², and X¹ is CR³,
when Q² and X² are both N, Q¹ is CR¹, and X¹ is CR³,
when Q¹ and Q² are both N, X¹ is CR³, and X² is CR⁴; and
wherein, in Formula (I)
Z is O or S, and
n is 0 or 1; and
wherein, in Formula (I)
Ar¹ and Ar² are independently unsubstituted or substituted aryl or heteroaryl, and the substituted aryl or heteroaryl is substituted by at least one substituent selected from a group consisting of halogen, cyano, NH₂, NHR⁵, NR⁶R⁷, NO₂, alkyl, OH, CF₃, —O— alkyl, —SO₂-alkyl, —SO₂N-alkyl, —S-alkyl and (C₁-C₄)alkoxy(C₁-C₄)alkyl,
wherein
R⁵ and R⁶ are independently alkyl, and R⁶ and R⁷ are the same, and
the alkyl can be unsubstituted or substituted straight-chain alkyl or branched-chain alkyl, and the substituted straight-chain alkyl or branched-chain alkyl is substituted by at least one substituent selected from a group consisting of halogen, oxygen and amino.

2. The small molecule compound or salt thereof or solvates of them as claimed in claim 1, wherein for R¹ and R², the alkyl is C₁-C₂₄ alkyl, and the C₁-C₂₄ alkyl is C₁-C₂₄ straight-chain alkyl or C₄-C₂₄ branched-chain alkyl.

3. The small molecule compound or salt thereof or solvates of them as claimed in claim 1, wherein for R¹ and R², the alkylhalo is C₁-C₂₄ alkylhalo, and the C₁-C₂₄ alkylhalo is C₁-C₂₄ straight-chain alkylhalo or C₄-C₂₄ branched-chain alkylhalo.

4. The small molecule compound or salt thereof or solvates of them as claimed in claim 3, wherein the alkylhalo is alkyl substituted by at least one halogen, and the at least one halogen is independently selected from a group consisting of F, Cl, Br and I.

5. The small molecule compound or salt thereof or solvates of them as claimed in claim 1, wherein for R¹ and R², the halogen is independently selected from a group consisting of F, Cl, Br and I.

6. The small molecule compound or salt thereof or solvates of them as claimed in claim 1, wherein for R³ and R⁴, the alkyl is C₁-C₂₄ alkyl, and the C₁-C₂₄ alkyl is C₁-C₂₄ straight-chain alkyl or C₄-C₂₄ branched-chain alkyl.

7. The small molecule compound or salt thereof or solvates of them as claimed in claim 1, wherein the Ar¹ and Ar² are independently selected from a group consisting of: 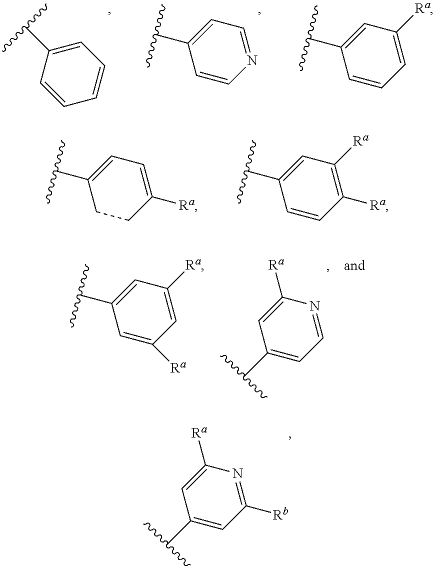
wherein R^{a} and R^{b} are independently selected from a group consisting of phenyl, C₁-C₆ alkyl, halogen, alkylhalo and cyano.

8. The small molecule compound or salt thereof or solvates of them as claimed in claim 7, wherein when R^{a} is halogen, it is independently selected from a group consisting of F, Cl, Br and I while when R^{b} is halogen, it is also independently selected from a group consisting of F, Cl, Br and I.

9. The small molecule compound or salt thereof or solvates of them as claimed in claim 7, wherein Ar¹ and Ar² are independently selected from a group consisting of:

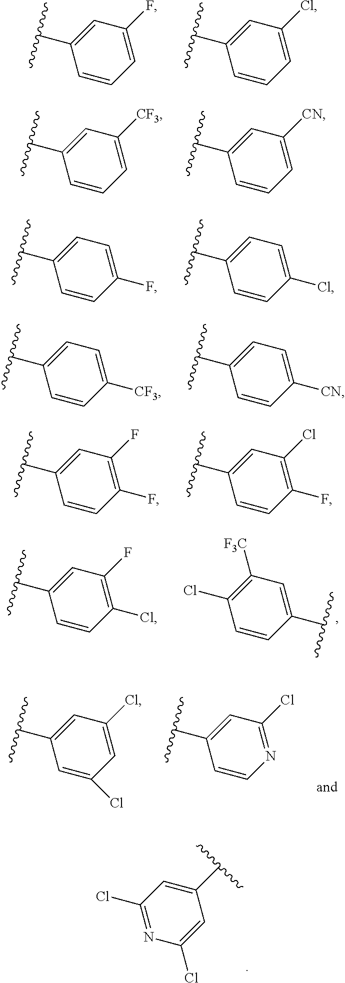

10. The small molecule compound or salt thereof or solvates of them as claimed in claim 1, wherein Q¹ and X² are both N, Q² is CR², and X¹ is CH.

11. The small molecule compound or salt thereof or solvates of them as claimed in claim 1, wherein Q¹ and Q² are both N, and X¹ and X² are both CH.

12. The small molecule compound or salt thereof or solvates of them as claimed in claim 1, wherein X² is N, Q¹ is CR¹, Q² is CR², and X¹ is CH.

13. The small molecule compound or salt thereof or solvates of them as claimed in claim 12, wherein R¹ and R² are both H.

14. The small molecule compound or salt thereof or solvates of them as claimed in claim 1, wherein the small molecule compound comprises: 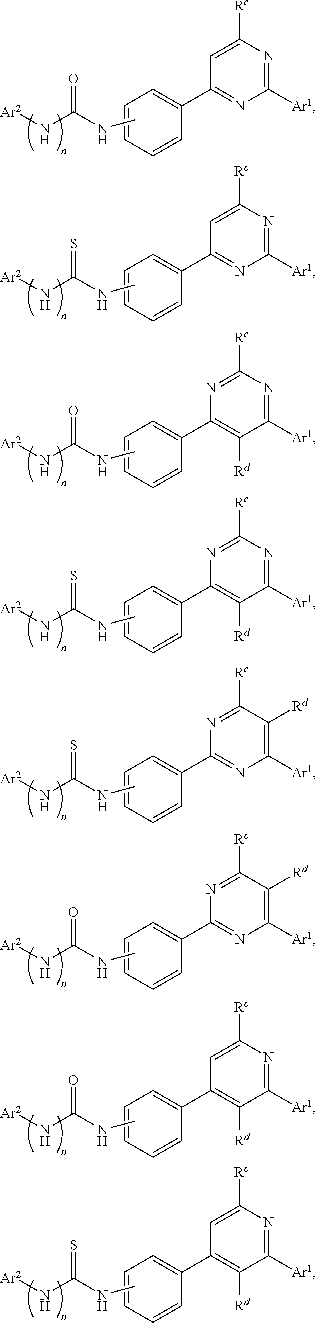 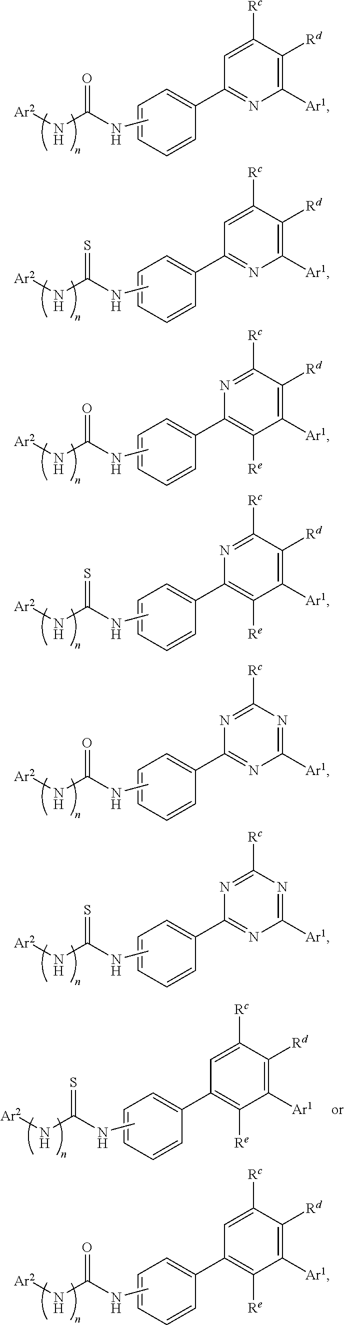
wherein R^{c}, R^{d} and R^{e} are independently H or alkyl.

15. The small molecule compound or salt thereof or solvates of them as claimed in claim 1, wherein the small molecule compound is selected from a group consisting of the following Compound 1 to Compound 15:

| Compound | |
|---|---|
| Number | Structure |
| 1 | 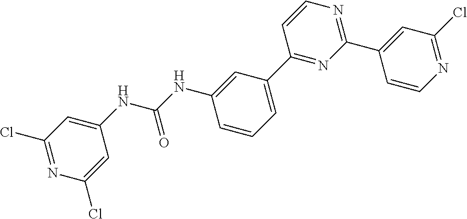 [SMILES: O=C(Nc1cccc(-c2ccnc(-c3ccnc(Cl)c3)n2)c1)Nc1cc(Cl)nc(Cl)c1] |
| 2 | 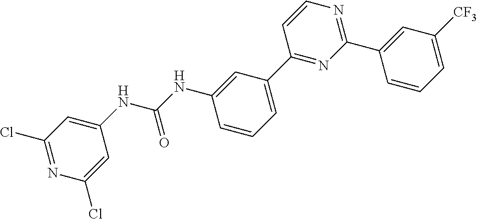 [SMILES: O=C(Nc1cccc(-c2ccnc(-c3cccc(C(F)(F)F)c3)n2)c1)Nc1cc(Cl)nc(Cl)c1] |
| 3 | 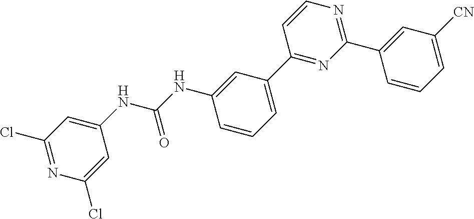 [SMILES: N#Cc1cccc(-c2nccc(-c3cccc(NC(=O)Nc4cc(Cl)nc(Cl)c4)c3)n2)c1] |
| 4 | 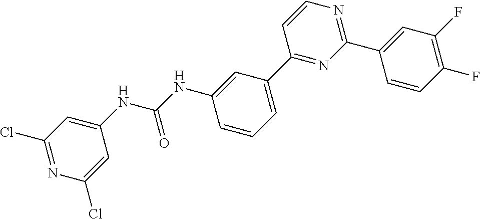 [SMILES: O=C(Nc1cccc(-c2ccnc(-c3ccc(F)c(F)c3)n2)c1)Nc1cc(Cl)nc(Cl)c1] |
| 5 | 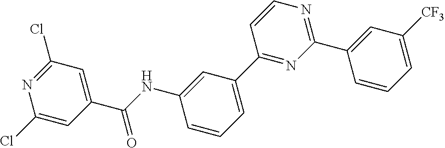 [SMILES: O=C(Nc1cccc(-c2ccnc(-c3cccc(C(F)(F)F)c3)n2)c1)c1cc(Cl)nc(Cl)c1] |
| 6 | 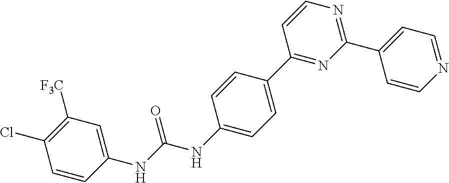 [SMILES: O=C(Nc1ccc(-c2ccnc(-c3ccncc3)n2)cc1)Nc1ccc(Cl)c(C(F)(F)F)c1] |
| 7 | 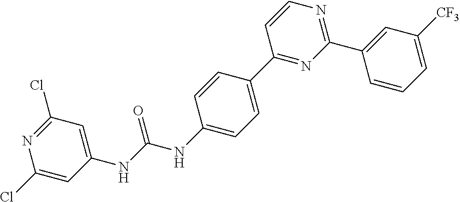 [SMILES: O=C(Nc1ccc(-c2ccnc(-c3cccc(C(F)(F)F)c3)n2)cc1)Nc1cc(Cl)nc(Cl)c1] |
| 8 | 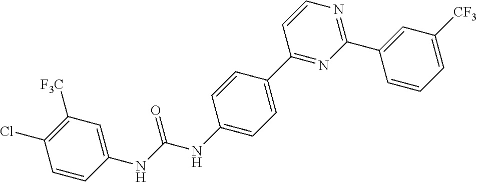 [SMILES: O=C(Nc1ccc(-c2ccnc(-c3cccc(C(F)(F)F)c3)n2)cc1)Nc1ccc(Cl)c(C(F)(F)F)c1] |
| 9 | 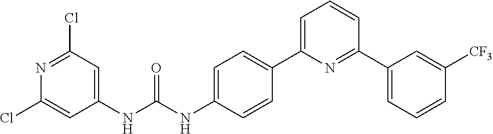 [SMILES: O=C(Nc1ccc(-c2cccc(-c3cccc(C(F)(F)F)c3)n2)cc1)Nc1cc(Cl)nc(Cl)c1] |
| 10 | 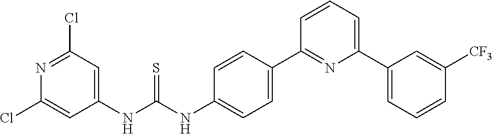 [SMILES: FC(F)(F)c1cccc(-c2cccc(-c3ccc(NC(=S)Nc4cc(Cl)nc(Cl)c4)cc3)n2)c1] |
| 11 | 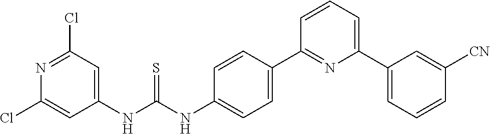 [SMILES: N#Cc1cccc(-c2cccc(-c3ccc(NC(=S)Nc4cc(Cl)nc(Cl)c4)cc3)n2)c1] |
| 12 | 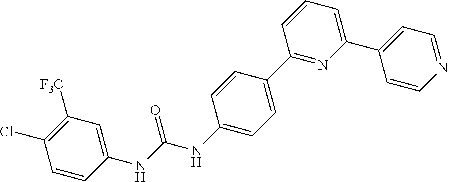 [SMILES: O=C(Nc1ccc(-c2cccc(-c3ccncc3)n2)cc1)Nc1ccc(Cl)c(C(F)(F)F)c1] |
| 13 | 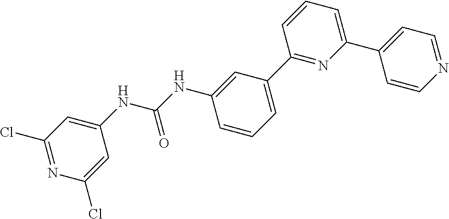 [SMILES: O=C(Nc1cccc(-c2cccc(-c3ccncc3)n2)c1)Nc1cc(Cl)nc(Cl)c1] |
| 14 | 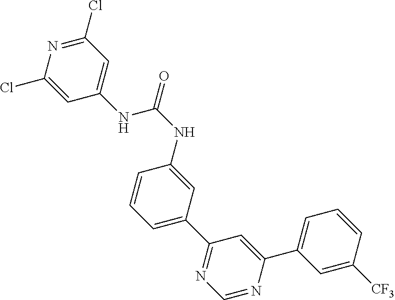 [SMILES: O=C(Nc1cccc(-c2cc(-c3cccc(C(F)(F)F)c3)ncn2)c1)Nc1cc(Cl)nc(Cl)c1] |
| 15 | 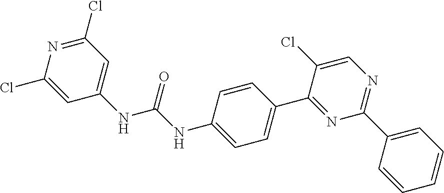 [SMILES: O=C(Nc1ccc(-c2nc(-c3ccccc3)ncc2Cl)cc1)Nc1cc(Cl)nc(Cl)c1] |

16. A guanine nucleotide-binding protein G(q) subunit alpha (GNAQ) inhibitor, comprising:
the small molecule compound or salt thereof or solvates of them as claimed in claim 1.

17. The guanine nucleotide-binding protein G(q) subunit alpha inhibitor as claimed in claim 16, wherein the small molecule compound is selected from a group consisting of the following Compound 1 to Compound 15:

| Compound | |
|---|---|
| Number | Structure |
| 1 | 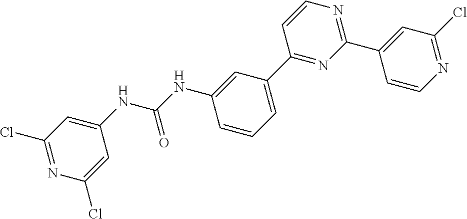 [SMILES: O=C(Nc1cccc(-c2ccnc(-c3ccnc(Cl)c3)n2)c1)Nc1cc(Cl)nc(Cl)c1] |
| 2 | 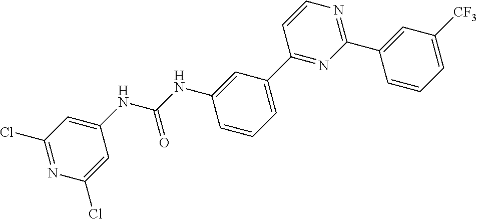 [SMILES: O=C(Nc1cccc(-c2ccnc(-c3cccc(C(F)(F)F)c3)n2)c1)Nc1cc(Cl)nc(Cl)c1] |
| 3 | 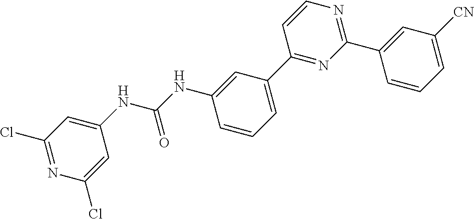 [SMILES: N#Cc1cccc(-c2nccc(-c3cccc(NC(=O)Nc4cc(Cl)nc(Cl)c4)c3)n2)c1] |
| 4 | 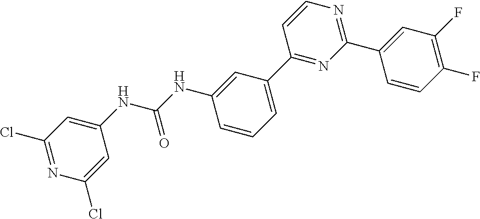 [SMILES: O=C(Nc1cccc(-c2ccnc(-c3ccc(F)c(F)c3)n2)c1)Nc1cc(Cl)nc(Cl)c1] |
| 5 | 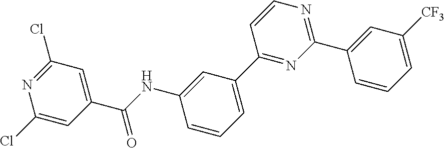 [SMILES: O=C(Nc1cccc(-c2ccnc(-c3cccc(C(F)(F)F)c3)n2)c1)c1cc(Cl)nc(Cl)c1] |
| 6 | 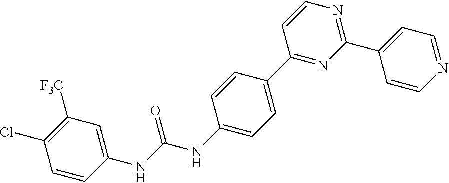 [SMILES: O=C(Nc1ccc(-c2ccnc(-c3ccncc3)n2)cc1)Nc1ccc(Cl)c(C(F)(F)F)c1] |
| 7 | 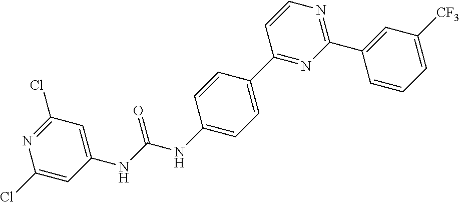 [SMILES: O=C(Nc1ccc(-c2ccnc(-c3cccc(C(F)(F)F)c3)n2)cc1)Nc1cc(Cl)nc(Cl)c1] |
| 8 | 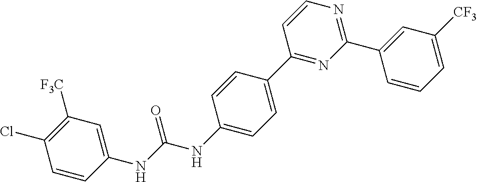 [SMILES: O=C(Nc1ccc(-c2ccnc(-c3cccc(C(F)(F)F)c3)n2)cc1)Nc1ccc(Cl)c(C(F)(F)F)c1] |
| 9 | 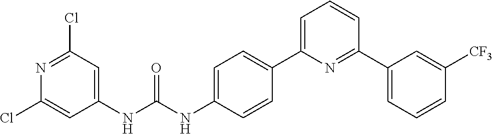 [SMILES: O=C(Nc1ccc(-c2cccc(-c3cccc(C(F)(F)F)c3)n2)cc1)Nc1cc(Cl)nc(Cl)c1] |
| 10 | 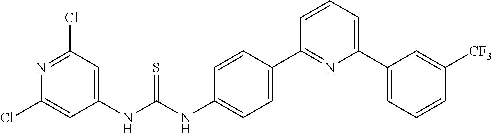 [SMILES: FC(F)(F)c1cccc(-c2cccc(-c3ccc(NC(=S)Nc4cc(Cl)nc(Cl)c4)cc3)n2)c1] |
| 11 | 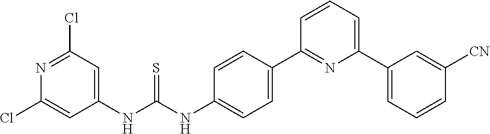 [SMILES: N#Cc1cccc(-c2cccc(-c3ccc(NC(=S)Nc4cc(Cl)nc(Cl)c4)cc3)n2)c1] |
| 12 | 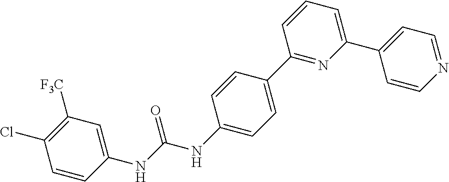 [SMILES: O=C(Nc1ccc(-c2cccc(-c3ccncc3)n2)cc1)Nc1ccc(Cl)c(C(F)(F)F)c1] |
| 13 | 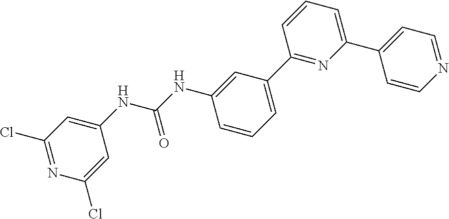 [SMILES: O=C(Nc1cccc(-c2cccc(-c3ccncc3)n2)c1)Nc1cc(Cl)nc(Cl)c1] |
| 14 | 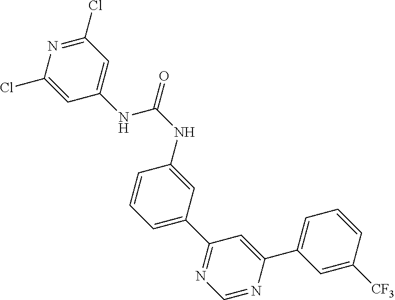 [SMILES: O=C(Nc1cccc(-c2cc(-c3cccc(C(F)(F)F)c3)ncn2)c1)Nc1cc(Cl)nc(Cl)c1] |
| 15 | 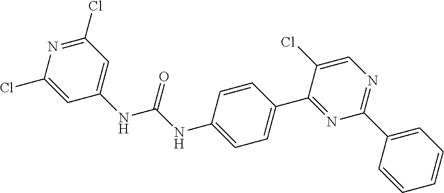 [SMILES: O=C(Nc1ccc(-c2nc(-c3ccccc3)ncc2Cl)cc1)Nc1cc(Cl)nc(Cl)c1] |

18. A method for treating and/or preventing a cancer, comprising:
administering the small molecule compound or salt thereof or solvates of them as claimed in claim 1 to a subject in need thereof.

19. The method for treating and/or preventing a cancer as claimed in claim 18, wherein the small molecule compound is selected from a group consisting of the following Compound 1 to Compound 15:

| Compound | |
|---|---|
| Number | Structure |
| 1 | 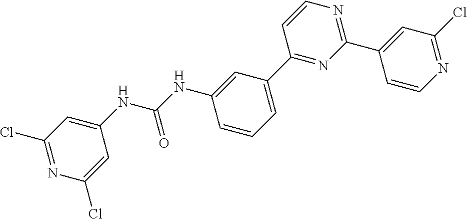 [SMILES: O=C(Nc1cccc(-c2ccnc(-c3ccnc(Cl)c3)n2)c1)Nc1cc(Cl)nc(Cl)c1] |
| 2 | 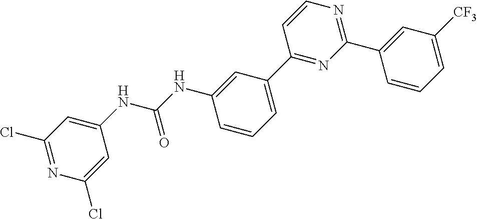 [SMILES: O=C(Nc1cccc(-c2ccnc(-c3cccc(C(F)(F)F)c3)n2)c1)Nc1cc(Cl)nc(Cl)c1] |
| 3 | 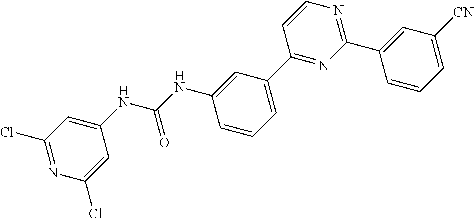 [SMILES: N#Cc1cccc(-c2nccc(-c3cccc(NC(=O)Nc4cc(Cl)nc(Cl)c4)c3)n2)c1] |
| 4 | 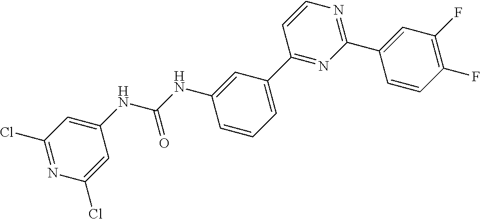 [SMILES: O=C(Nc1cccc(-c2ccnc(-c3ccc(F)c(F)c3)n2)c1)Nc1cc(Cl)nc(Cl)c1] |
| 5 | 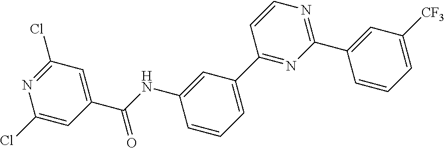 [SMILES: O=C(Nc1cccc(-c2ccnc(-c3cccc(C(F)(F)F)c3)n2)c1)c1cc(Cl)nc(Cl)c1] |
| 6 | 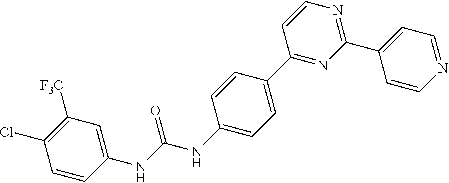 [SMILES: O=C(Nc1ccc(-c2ccnc(-c3ccncc3)n2)cc1)Nc1ccc(Cl)c(C(F)(F)F)c1] |
| 7 | 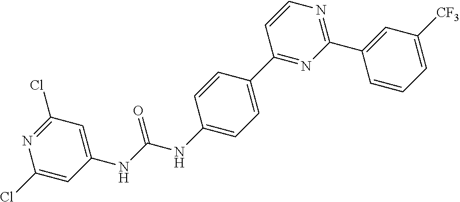 [SMILES: O=C(Nc1ccc(-c2ccnc(-c3cccc(C(F)(F)F)c3)n2)cc1)Nc1cc(Cl)nc(Cl)c1] |
| 8 | 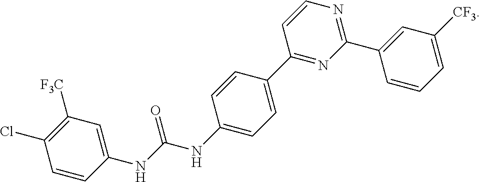 [SMILES: O=C(Nc1ccc(-c2ccnc(-c3cccc(C(F)(F)F)c3)n2)cc1)Nc1ccc(Cl)c(C(F)(F)F)c1] |
| 9 | 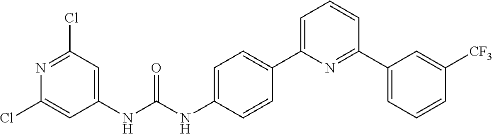 [SMILES: O=C(Nc1ccc(-c2cccc(-c3cccc(C(F)(F)F)c3)n2)cc1)Nc1cc(Cl)nc(Cl)c1] |
| 10 | 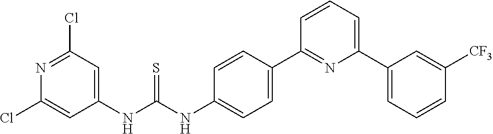 [SMILES: FC(F)(F)c1cccc(-c2cccc(-c3ccc(NC(=S)Nc4cc(Cl)nc(Cl)c4)cc3)n2)c1] |
| 11 | 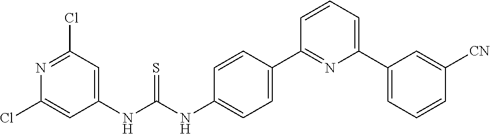 [SMILES: N#Cc1cccc(-c2cccc(-c3ccc(NC(=S)Nc4cc(Cl)nc(Cl)c4)cc3)n2)c1] |
| 12 | 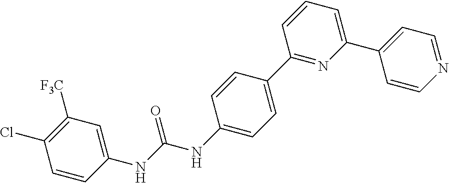 [SMILES: O=C(Nc1ccc(-c2cccc(-c3ccncc3)n2)cc1)Nc1ccc(Cl)c(C(F)(F)F)c1] |
| 13 | 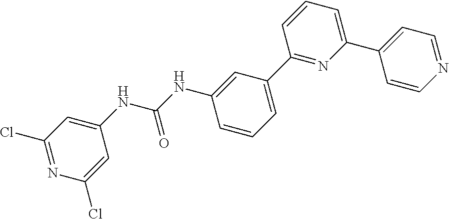 [SMILES: O=C(Nc1cccc(-c2cccc(-c3ccncc3)n2)c1)Nc1cc(Cl)nc(Cl)c1] |
| 14 | 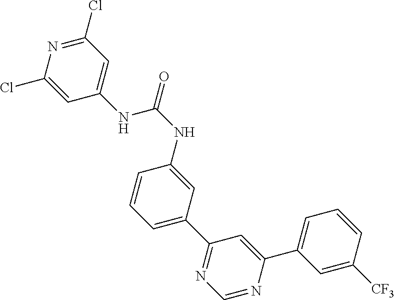 [SMILES: O=C(Nc1cccc(-c2cc(-c3cccc(C(F)(F)F)c3)ncn2)c1)Nc1cc(Cl)nc(Cl)c1] |
| 15 | 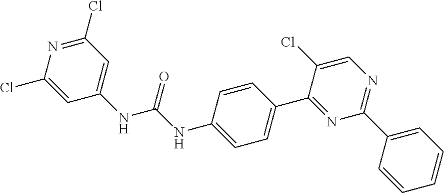 [SMILES: O=C(Nc1ccc(-c2nc(-c3ccccc3)ncc2Cl)cc1)Nc1cc(Cl)nc(Cl)c1] |

20. The method for treating and/or preventing a cancer as claimed in claim 18, wherein the cancer comprises melanoma, lymphoma, liver cancer, or breast cancer.
